SEQUENCE LISTING (6 sequences)

SEQ ID NO: 1 - DNA, 20 bp, synthetic construct
  source 1..20: mol_type = other DNA
  misc_feature 1..20: function = EPCAM qPCR F
gctggccgtaaactgctttg

SEQ ID NO: 2 - DNA, 20 bp, synthetic construct
  source 1..20: mol_type = other DNA
  misc_feature 1..20: function = EPCAM qPCR R
acatttggcagccagctttg

SEQ ID NO: 3 - DNA, 20 bp, synthetic construct
  source 1..20: mol_type = other DNA
  misc_feature 1..20: function = TTC3 qPCR F
tgtccctgtgaggattgcac

SEQ ID NO: 4 - DNA, 20 bp, synthetic construct
  source 1..20: mol_type = other DNA
  misc_feature 1..20: function = TTC3 qPCR R
ttgagcacacgcacgttttt

SEQ ID NO: 5 - DNA, 20 bp, synthetic construct
  source 1..20: mol_type = other DNA
  misc_feature 1..20: function = H4C5 qPCR F
gcggctattttcgttggtgt

SEQ ID NO: 6 - DNA, 20 bp, synthetic construct
  source 1..20: mol_type = other DNA
  misc_feature 1..20: function = H4C5 qPCR R
tgccctggatgttatctcgc

(19) United States
(12) Patent Application Publication
(10) Pub. No.: US 2025/0051852 A1
(43) Pub. Date: Feb. 13, 2025

(54) BIOMARKERS TO DETECT AGGRESSIVE PROSTATE CANCER FROM INDOLENT FORMS AND TREATMENT THEREOF

(71) Applicant: The Johns Hopkins University, Baltimore, MD (US)

(72) Inventor: Ranjan J. Perera, Orlando, FL (US)

(73) Assignee: The Johns Hopkins University, Baltimore, MD (US)

(21) Appl. No.: 18/718,373

(22) PCT Filed: Dec. 12, 2022
(86) PCT No.: PCT/US22/81340

§ 371 Date: Jun. 10, 2024

Related U.S. Application Data
(60) Provisional application No. 63/288,157, filed on Dec. 10, 2021.

(51) Int. Cl. C12Q 1/6886; G01N 33/574

(52) U.S. Cl. CPC C12Q 1/6886; G01N 33/57434; C12Q 2600/112; C12Q 2600/158; C12Q 2600/178

(57) ABSTRACT

The present invention relates to the field of cancer. More specifically, the present invention provides methods and compositions useful for detecting and treating prostate cancer. In a specific embodiment, a method for identifying a patient as having aggressive prostate cancer comprising the step of detecting overexpression relative to a control of epithelial cell adhesion molecule (EpCAM), H4 clustered histone 5 (H4C5), and tetratricopeptide repeat domain 3 (TTC3) in a urine sample obtained from the patient. In a more specific embodiment, the detecting step comprises detecting protein or ribonucleic acid (RNA) level of EPCAM, H4C5 and TTC3. In another specific embodiment, the method further comprises detecting overexpression relative to a control of one or more of messenger ribonucleic acid (mRNA), circulating RNA (circRNA), extracellular DNA and long non-coding RNA (lncRNA). The method can further comprise detecting metabolites.

### CROSS-REFERENCE TO RELATED APPLICATIONS

This application claims the benefit of U.S. Provisional Application No. 63/288,157, filed Dec. 10, 2021, which is incorporated herein by reference in its entirety.

### FIELD OF THE INVENTION

The present invention relates to the field of cancer. More specifically, the present invention provides methods and compositions useful for detecting and treating prostate cancer.

### REFERENCE TO AN ELECTRONIC SEQUENCE LISTING

The text of the computer readable sequence listing filed herewith, titled “P17007-02”, created Dec. 12, 2022, having a file size of 8,614 bytes, is hereby incorporated by reference in its entirety.

### BACKGROUND OF THE INVENTION

Prostate Cancer (PCa) is one of the leading causes of cancer deaths among American men. According to the National Cancer Institute estimates, 249,000 new prostate cancer cases will be diagnosed, and approximately 34,000 men will die from this disease in 2021. The estimated number of diagnoses represents a small fraction of disease-related biopsies performed each year. Prostate-Specific Antigen (PSA) test is a widely used test for screening men for this cancer. However, PSA cannot differentiate aggressive cancer from nonaggressive form and its high false-positive rate, unsubstantiated outcome, and small benefit justify an urgent unmet need for novel and more accurate diagnostic biomarkers for prostate cancer detection and to differentiate aggressive cancer from its indolent form.

### SUMMARY OF THE INVENTION

In one aspect, the present invention provides compositions and methods useful for identifying a patient as having aggressive prostate cancer. In particular embodiments, the present invention is useful for distinguishing among aggressive prostate cancer, indolent prostate cancer, benign prostrate hyperplasia (BPH) and prostatitis (PTT).

In some embodiments, a method for identifying a patient as having aggressive prostate cancer comprises the step of detecting overexpression relative to a control of epithelial cell adhesion molecule (EpCAM), H4 clustered histone 5 (H4C5), and tetratricopeptide repeat domain 3 (TTC3) in a sample obtained from the patient.

In specific embodiments, the detecting step comprises detecting protein level of EpCAM in a urine sample. In other specific embodiments, the detecting step comprises detecting ribonucleic acid (RNA) level of H4C5 and TTC3. In particular embodiments, the detecting step is performed using polymerase chain reaction (PCR).

In certain embodiments, the method distinguishes among aggressive prostate cancer, indolent prostate cancer, benign prostate hyperplasia and prostatitis.

In additional embodiments, the method for identifying a patient as having aggressive prostate cancer further comprises detecting overexpression relative to a control of one or more of messenger ribonucleic acid (mRNA), circulating RNA (circRNA), extracellular DNA and long non-coding RNA (lncRNA).

In some embodiments, the mRNA comprises one or more of RIDA, H1-4, H4C2 and H4C3. In certain embodiments, the circRNA comprises one or more of circ842, circ3266, circ1809, circ1979, circ645 and circ1607. In particular embodiments, the lncRNA comprises Inc-CCDC125-13 and/or ZNF667-AS1.

In some embodiments, the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

In certain embodiments, the method for identifying a patient as having aggressive prostate cancer further comprises detecting one or more metabolites selected from the group consisting of asparagine, aspartate, glycerate, citrate, isocitrate, glutamate, itaconate, malate, meglutol, cis-aconitate, isoleucine, leucine, pantothenate, glutamine, nicotinate, threonine, ketoglutarate, alpha-ketoisovaleric acid (KIVA), cysteine, 3P glycerate, xanthine and hypoxanthine.

In some embodiments, the method for identifying a patient as having aggressive prostate cancer further comprises the step of administering a prostate cancer therapy to the patient identified as having aggressive prostate cancer. In particular embodiments, the prostate cancer therapy comprises prostatectomy, radiation therapy, cryotherapy, hormone therapy, chemotherapy, immunotherapy and combinations thereof. Further examples of specific treatments are described herein.

In another aspect, the present invention provides methods for treating a patient having aggressive prostate cancer comprising the step of administering a prostate cancer therapy to a patient identified as having overexpression of EPCAM, H4C5 and TTC3 in a sample relative to a control. In some embodiments, the patient sample further comprises overexpression relative to a control of one or more of mRNA, circRNA, eccDNA and lncRNA.

In some embodiments, the mRNA comprises one or more of RIDA, H1-4, H4C2 and H4C3. In certain embodiments, the circRNA comprises one or more of circ842, circ3266, circ1809, circ1979, circ645 and circ1607. In particular embodiments, the lncRNA comprises Inc-CCDC125-13 and/or ZNF667-AS1.

In some embodiments, the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

In another aspect, the present invention provides a method for identifying a patient as having aggressive prostate cancer comprising the step of detecting the overexpression of one or more of protein, mRNA, circRNA, eccDNA lncRNA in a sample obtained from the patient relative to a control. In some embodiments, the protein comprises EPCAM. In some embodiments, the mRNA comprises one or more of H4C5, TTC3, RIDA, H1-4, H4C2 and H4C3. In particular embodiments, the circRNA comprises one or more of circ842, circ3266, circ1809, circ1979, circ645 and circ1607. In certain embodiments, the lncRNA comprises Inc-CCDC125-13 and/or ZNF667-AS1.

In some embodiments, the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

In some embodiments, the detecting step of the methods described herein utilizes a lateral flow device. In a specific embodiments, the lateral flow device comprises a dipstick assay.

In some embodiments, the sample comprises free-flow urine and/or prostate massaged urine. In some embodiments, the sample comprises blood or serum. In particular embodiments, the detection of H4C5 and TTC3 RNA comprises detection in blood or serum.

In specific embodiments, RNA markers are detected using polymerase chain reaction (PCR). In more specific embodiments, the PCR is qPCR. In embodiments, in which EPCAM, TTC3 and/or H4C5 are detected via PCR, primers can include SEQ ID NOS:1-6, respectively.

In further embodiments, the decreased expression relative to a control of one or more of the following can be used in the methods of the present invention: COX20, CAPN3, CANX, PBLD, LUC7L3, HMGN2P5, SNURF, PNPO, NUDT4, AK4, RSL1D1, UGDH, TRAPPC5, ZNF181, NPM1, PTMA, VDAC1, HSPD1, HSPE1, NIT2, RBIS, COX6C, ODC1, DDAH1, MRPL51, GATD3B, COA4, ATP5MC1, MAP7, HOMER2, NFIX, CCDC58, and COX5A.

In some embodiments, the increased (over) expression relative to a control of one or more of the following can be used: TTC3, ZNF91, H4C2, EEF1G, TOM1L1, H4C3, ELK4, H1-4, OST4, H4C5, RIDA, MRPS21, NCALD, NDUFB9, RAN, EPCAM, and TMEM263.

In particular embodiments, EPCAM protein and H4C5 and TTC RNA can be measured along with one or more of the other markers described herein.

### BRIEF DESCRIPTION OF THE FIGURES

FIG. 1A-1D. Identification and validation of the urine-enriched liquid biopsy biomarkers in PCa. FIG. 1A: Flow chart for the identification of the urine-enriched liquid biopsy biomarkers in PCa. FIG. 1B: Top 50 mRNAs with the highest expression in primary and metastasis of PCa compared with the normal from TCGA database. Red and blue indicate upregulated and downregulated genes, respectively. FIG. 1C: qPCR analysis showing the distribution of normalized expression values of the 50 mRNAs in pooled PCa and normal urine. Values indicate fold change relative to pooled normal urine. FIG. 1D: The differential expression of 9 mRNAs in 20 PCa and normal urine was validated by qPCR. N, normal; PCa, prostate cancer; qPCR, quantitative reverse transcription PCR.

FIG. 2A-2C. Urinary EPCAM protein, TTC3 and H4C5 RNA levels in PCa. FIG. 2A: Scatter plots representing urinary EPCAM protein concentrations for each normal or PCa patient determined by ELISA. The EPCAM protein was detected pre- and post-prostatectomy in each PCa patient. FIG. 2B: Scatter plots representing urinary TTC3 RNA levels for each normal or PCa patient determined by qPCR. The TTC3 RNA was detected pre- and post-prostatectomy in each PCa patient. FIG. 2C: Scatter plots representing urinary H4C5 RNA levels for each normal or PCa patient determined by qPCR. The H4C5 RNA was detected pre- and post-prostatectomy in each PCa patient.

FIG. 3. Urinary EPCAM protein, TTC3 and H4C5 RNA are potential biomarkers for PCa diagnosis. Receiver operating characteristic curve (ROC) for urinary EPCAM protein, TTC3 and H4C5 RNA levels in patients with PCa versus control urine. The area under the curve (AUC) is shown for each ROC analysis, making 0.99 for EPCAM protein, 0.96 for H4C5 RNA, and 0.92 for TTC3 RNA. N, normal; PCa, prostate cancer; ELISA, enzyme-linked immunosorbent assay; qPCR, quantitative reverse transcription PCR.

FIG. 4A-4D. TTC3 silencing inhibits the growth and invasion of PCa cells in vitro. FIG. 4A: Transcripts expression of TTC3 in human PCa (PC3, LNCaP) and normal prostate epithelial (HPrEC) cell lines was detected by qPCR. FIG. 4B: siRNA-mediated depletion of TTC3 was determined by qPCR. FIG. 4C: The effect of TTC3-specific siRNA on the proliferation of PCa cells by MTS. FIG. 4D: Representative images of PC3 and LNCaP invasion of cells treated with TTC3 siRNA on the membrane. Data, mean±SEM. *, P<0.05, **, P<0.01.

FIG. 5A-5C. Expression of FDA-approved diagnostic markers urinary prostate cancer antigen 3 (PCA3) and serum prostate-specific antigen (PSA) in PCa. FIG. 5A: Scatter plots representing urinary PCA3 RNA levels for each normal or PCa patient determined by qPCR. The PCA3 RNA was detected pre- and post-prostatectomy in each PCa patient. FIG. 5B: Scatter plots representing urinary SPDEF RNA levels for each normal or PCa patient determined by qPCR. The SPDEF RNA was detected pre- and post-prostatectomy in each PCa patient. FIG. 5C: The serum PSA protein was detected pre- and post-prostatectomy in each PCa patient.

FIG. 6A-6B. Waterfall Plot of the urinary biomarker expression in PCa and normal. FIG. 6A: Each bar represents an individual sample's mean value, increasing left to right. The black horizontal line on each plot indicates a cutoff value of 26.86 pg/ml for EPCAM protein, 69.92 for TTC3 RNA, and 1068.32 for H4C5 RNA. FIG. 6B: The black horizontal line on each plot indicates a cutoff value of 5.69 for PCA3 RNA, and 32.76 for SPDEF RNA. The horizontal gray bar indicates the number of patients misdiagnosed as positive or negative with the cut point. N, normal; PCa, prostate cancer; ELISA, enzyme-linked immunosorbent assay; qPCR, quantitative reverse transcription PCR.

FIG. 7A-7D. EPCAM silencing inhibits the growth and invasion of PCa cells in vitro. FIG. 7A: Transcripts expression of EPCAM in human PCa (PC3, LNCaP) and normal prostate epithelial (HPrEC) cell lines was detected by qPCR. FIG. 7B: siRNA-mediated depletion of EPCAM was determined by qPCR. FIG. 7C: The effect of EPCAM-specific siRNA on the proliferation of PCa cells by MTS. FIG. 7D: Representative images of PC3 and LNCaP invasion of cells treated with EPCAM siRNA on the membrane. Data, mean±SEM. *P<0.05, **, P<0.01.

FIG. 8A-8B. Identification of PCa-specific mRNAs in urine. FIG. 8A: Principle component analysis (PCA) results depict the separation of normal and tumor samples of RNA-seq data. FIG. 8B: Fifty-one gene panel separates control, metastasis and primary tumors.

FIG. 9A-9B. Confirmation of selected targets by qPCR. FIG. 9A: Contribution of comparisons 1 and 2. Control compared to metastasis and control compared to primary tumors in the TCGA dataset. FIG. 9B: qPCR validation of candidate genes.

FIG. 10. EPCAM, TTC3 and PCA3 gene expression in a panel of normal, BPH, Prostatitis and PCa urine samples. 20 normal, 11 BPH, 7 prostatisis and 25 PCa urine samples were tested. Both TTC3 and EPCAM clearly separate PCa from other groups.

FIG. 11A-11D. Principle component analysis of clusters (delta Ct values) of four groups. FIG. 11A: EPCAM+TTC3+PCA. FIG. 11B: PCA3+TTC3. FIG. 11C: EPCAM+PCA3. FIG. 11D: EPCAM+TTC3. EPCAM+TTC3 shows the best separate of prostate cancer from the other groups.

FIG. 12A-12B. Prostate cancer specific circular RNAs in urine. FIG. 12A: Volcano plot depict the differentially expressed circRNAs in PCa urine compared to normal. FIG. 12B: Highly upregulated circRNAs in PCa urine compared to normal.

FIG. 13. Significance test of markers. PCA could not distinguish BPH/PTT (Prostatitis) v. PCa (non-significant P-value). The highest significance comparison in the dataset is shown in yellow.

FIG. 14. Significance test of markers. The % of sensitivity of EPCAM and TTC3 show the highest sensitivity among the three tested markers.

FIG. 15. A novel group of eccDNAs specific for PCa. eccDNAs were identified using a previously published DNA-seq data. Some of these candidates are validated (ongoing) in DNA samples in PCa patient samples.

FIG. 16A-16B. EPCAM ELISA test. Commercial ELISA assay kit for EPCAM was used and developed the test to measure EPCAM mRNA levels in the urine. EPCAM expression in 20 normal (N), 17 prostate cancer (PCa) patient urine samples.

FIG. 17. Inc-RNA expression in PCa urine. PCA decomposition of total RNA.

FIG. 18. Volcano plot of lncRNAs only. Total 53 significant lncRNAs (Incipedia annotation). Only two are upregulated in PCa. *(lfc2)>1 and adjPval<0.05.

FIG. 19A-19B. DE lncRNAs PCa/Normal.

FIG. 20A-20B. PCA (FIG. 13A) and heatmap (FIG. 13B) plot of 53 lncRNAs.

FIG. 21A-21F. Expression of top DE lncRNAs. ENTPD1-AS1 (FIG. 14A); LINC01973 (FIG. 14B); LINC02312 (FIG. 14C); CPB2-AS1 (FIG. 14D); lnc-CCDC125-13 (FIG. 14E); and ZNF667-AS1 (FIG. 14F).

FIG. 22. EPCAM ELISA for PCa. Normal 46, PCa before-operative 49, PCA after-operative 24.

FIG. 23. Metabolites high in PCa v. Normal.

FIG. 24. Metabolites high in PCa v. BPH.

FIG. 25. Metabolites up in PCa v. PTT.

FIG. 26. Heatmap for significant metabolies.

FIG. 27. Meabolites down in PCa v. PTT.

FIG. 28. The receiver operating characteristics curve (ROC) analysis of biomarkers. A series of cutoff points are illustrated as black dots.

FIG. 29. ROC Curve Comparison of EPCAM, H4C5 and TTC3.

FIG. 30. EPCAM expression is higher in PCa urine than normal urine-ELISA assay.

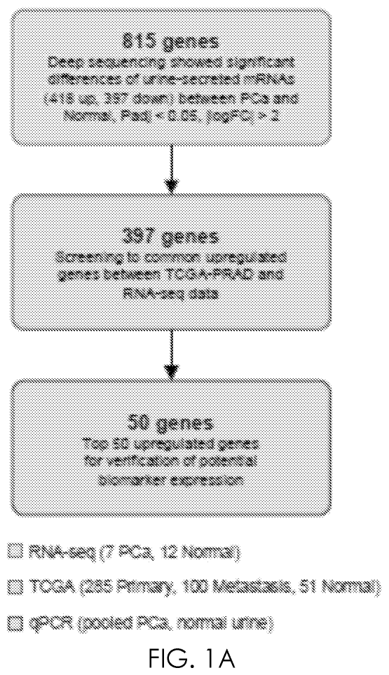

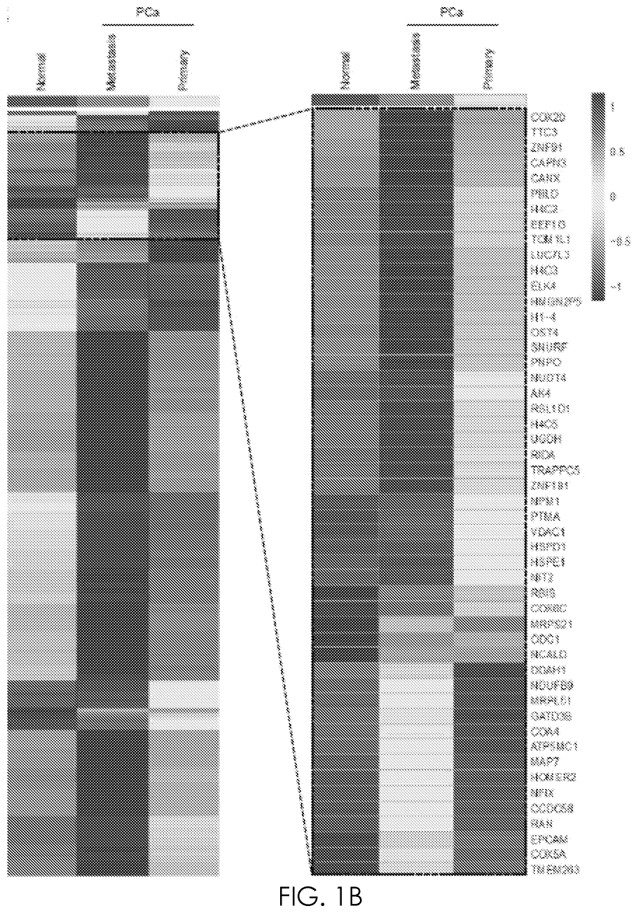

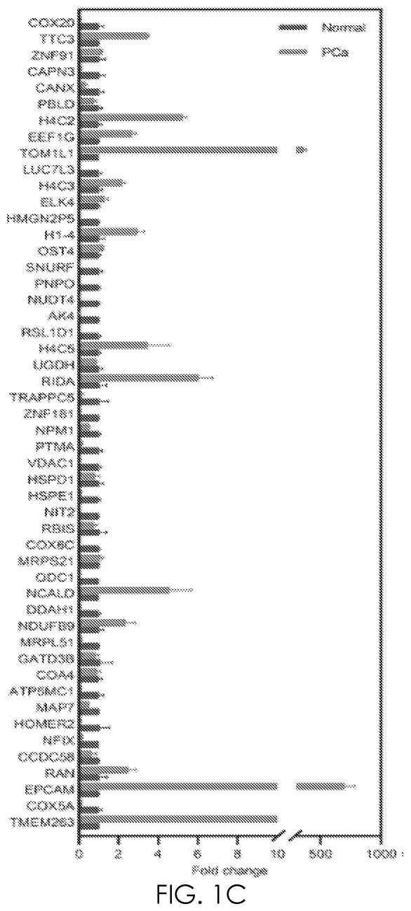

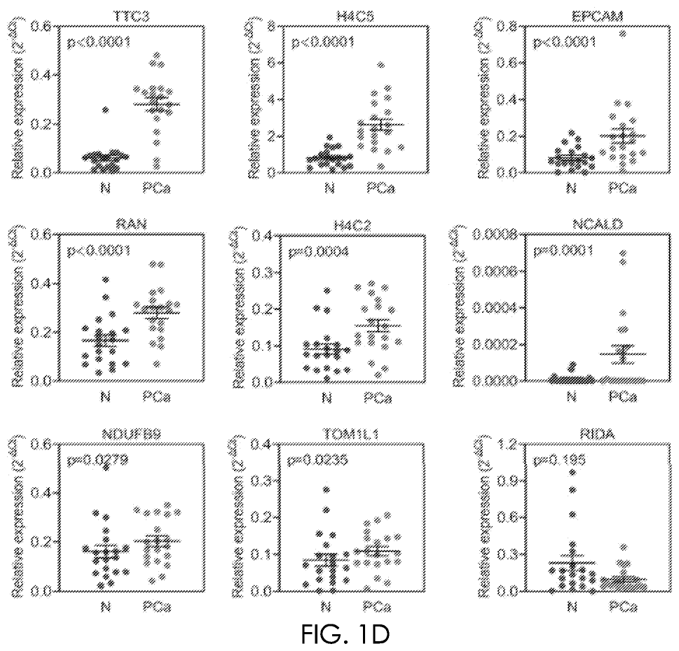

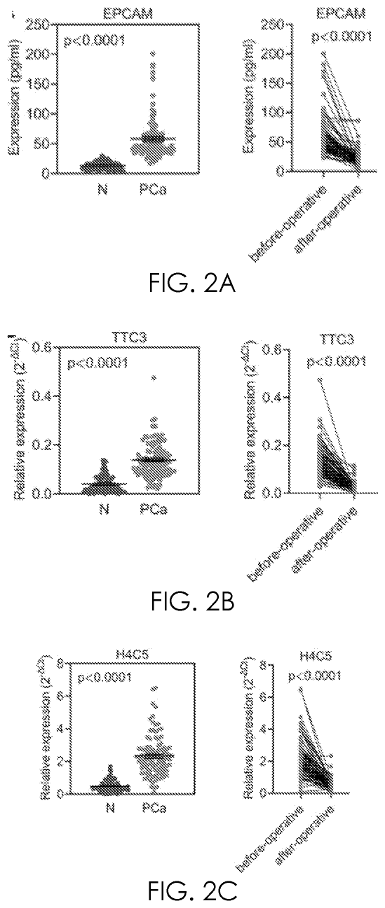

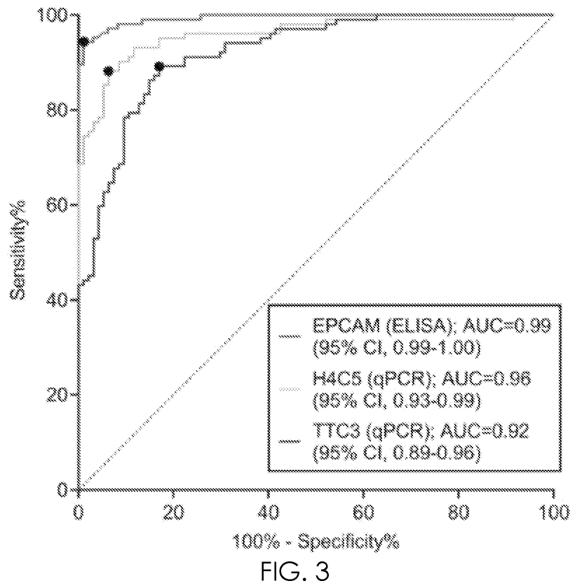

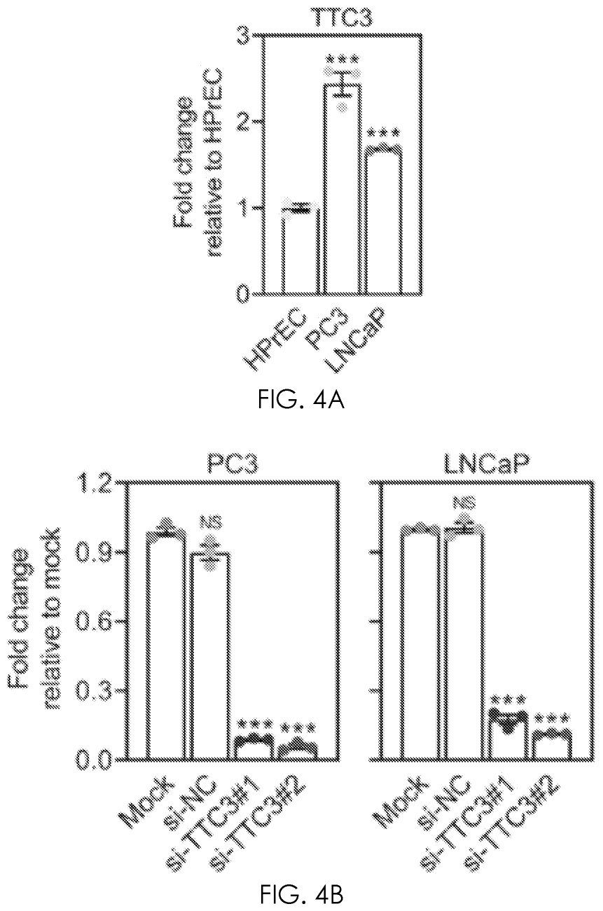

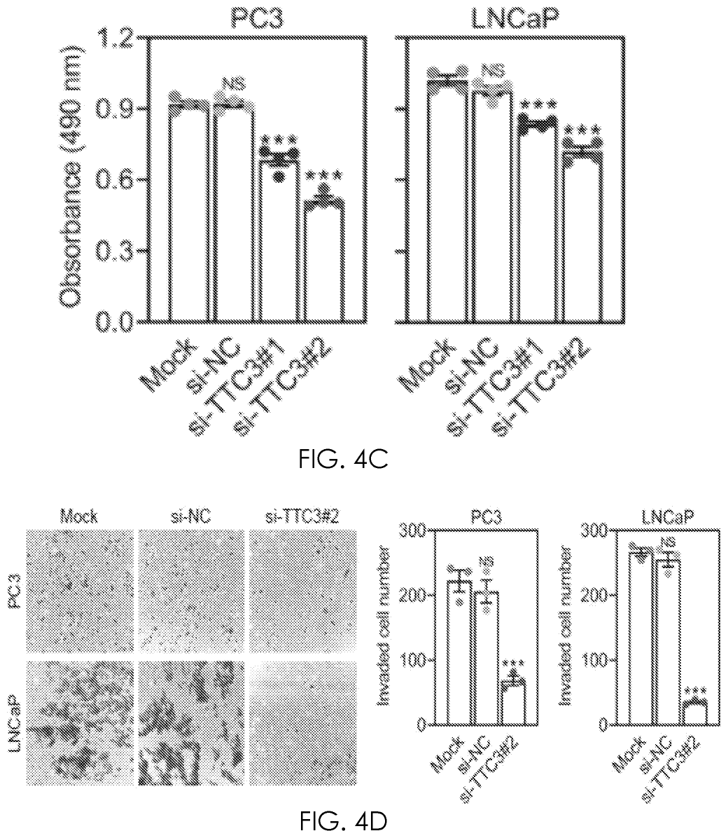

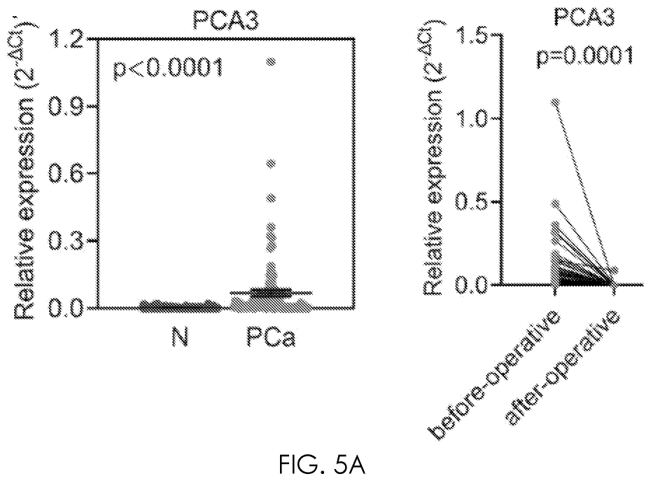

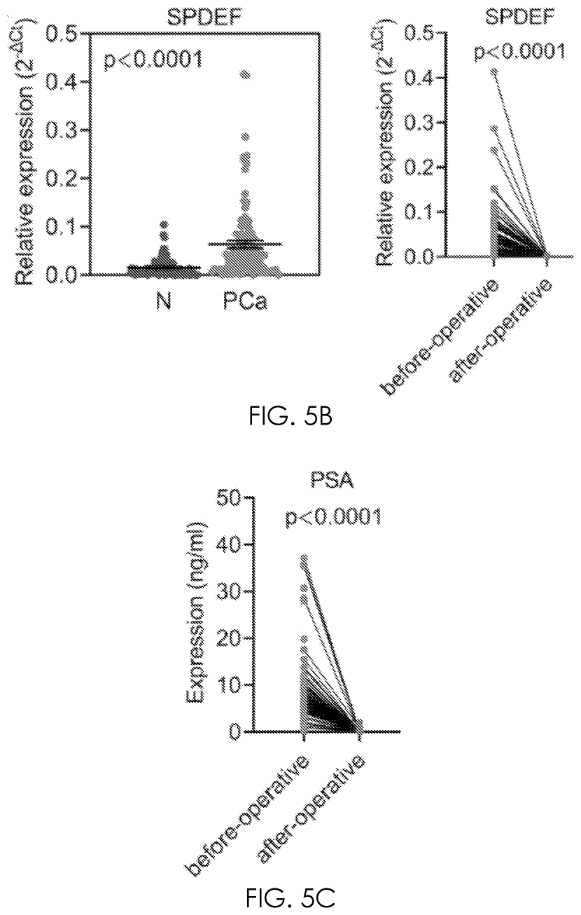

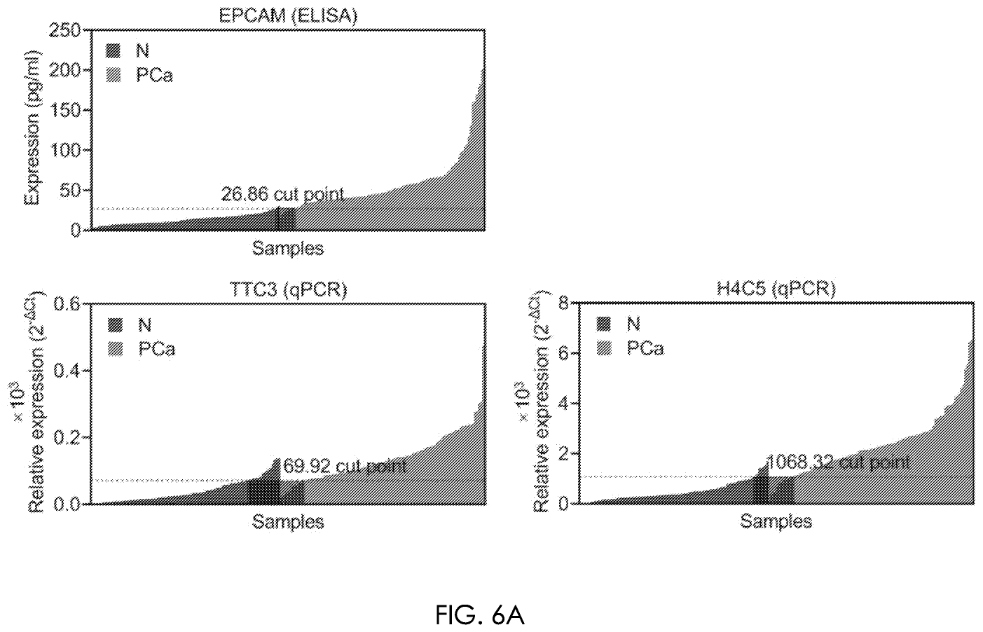

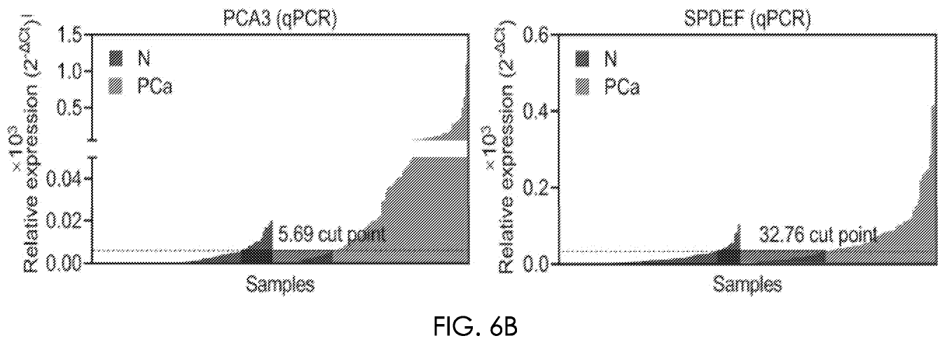

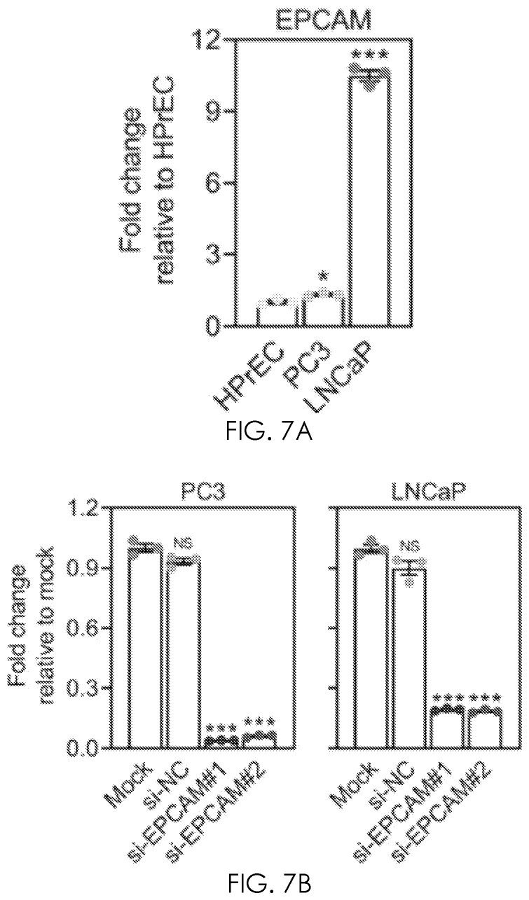

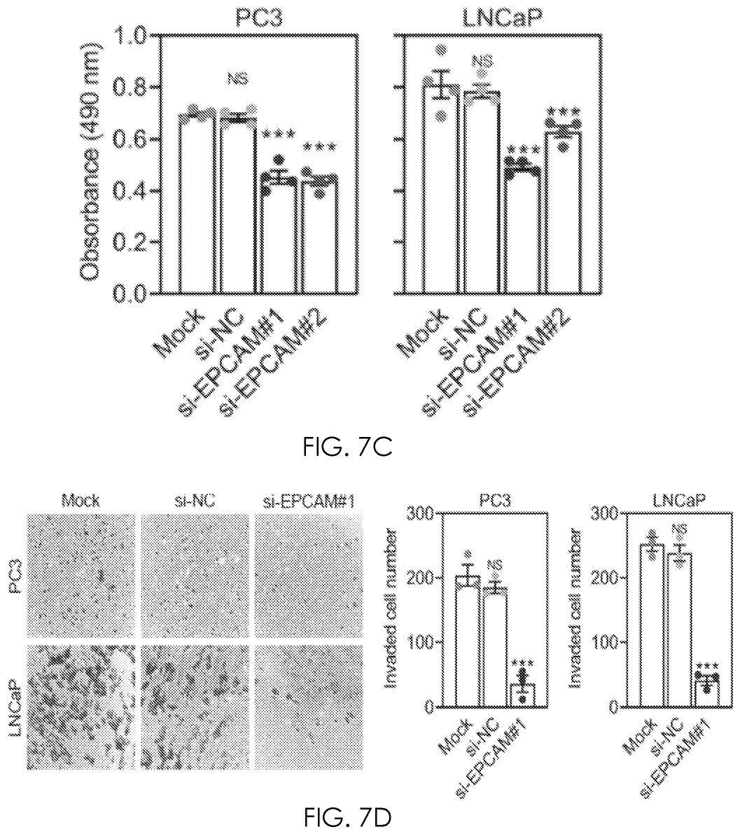

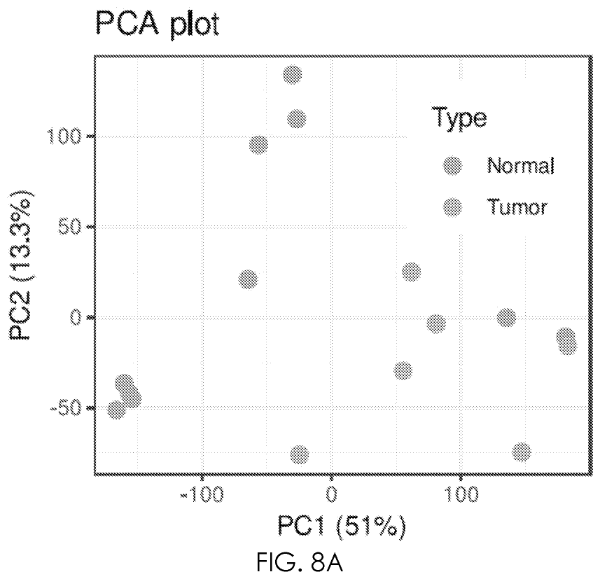

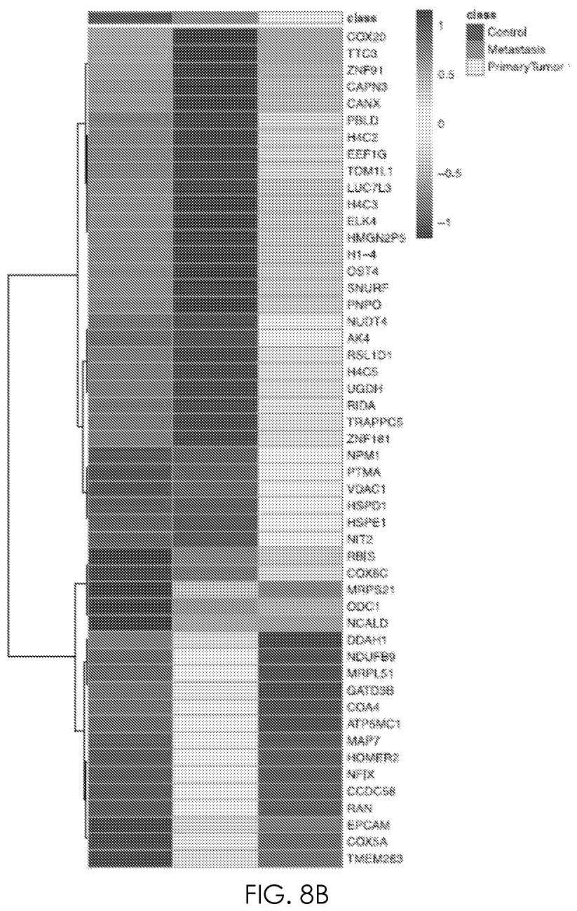

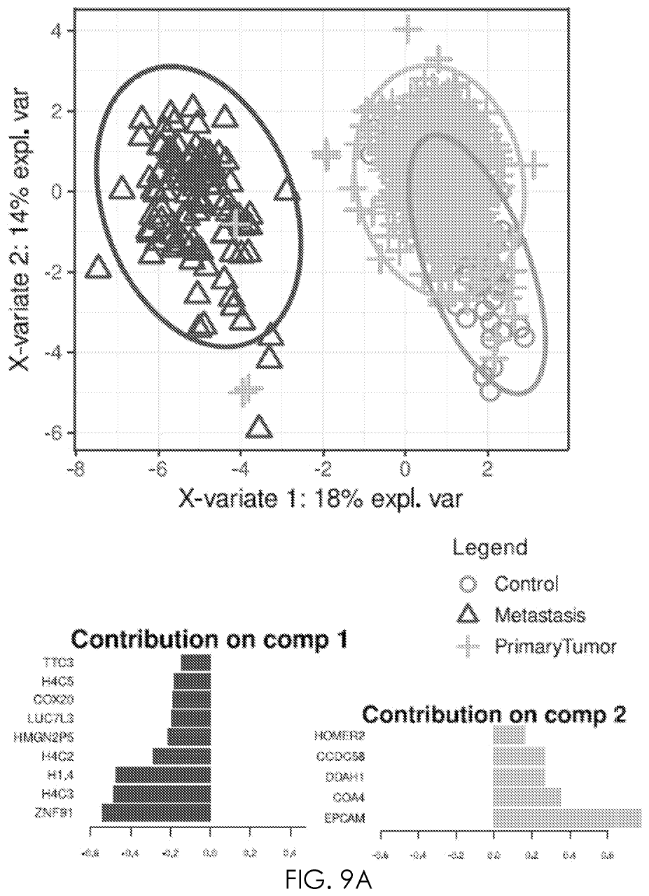

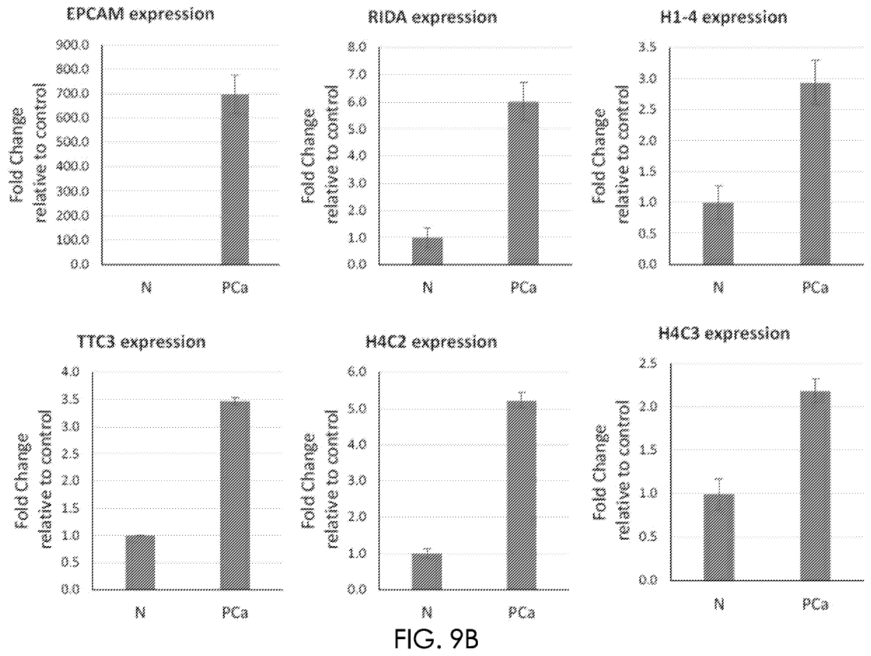

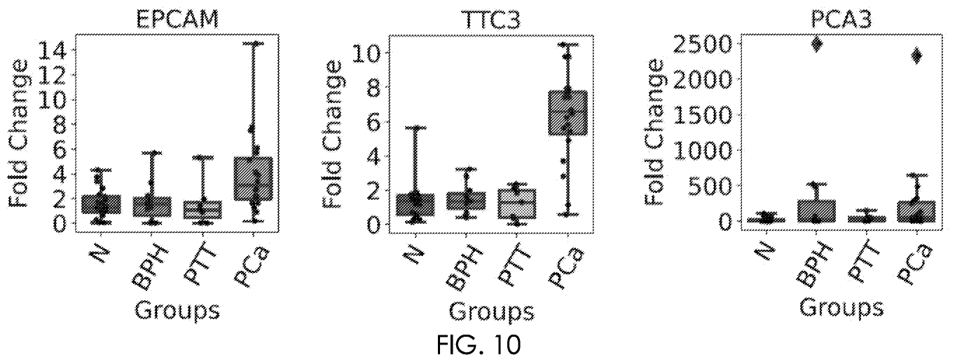

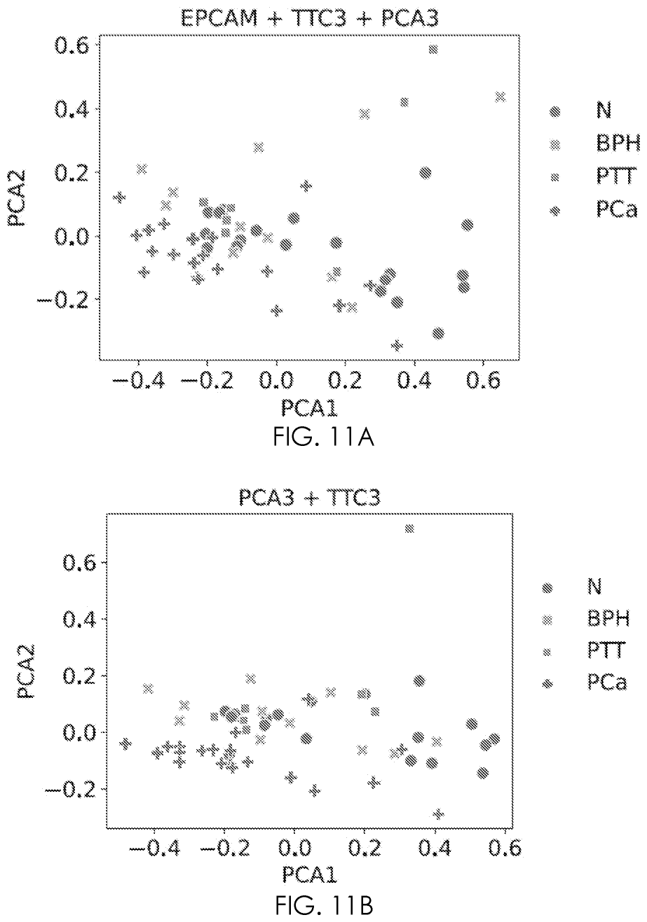

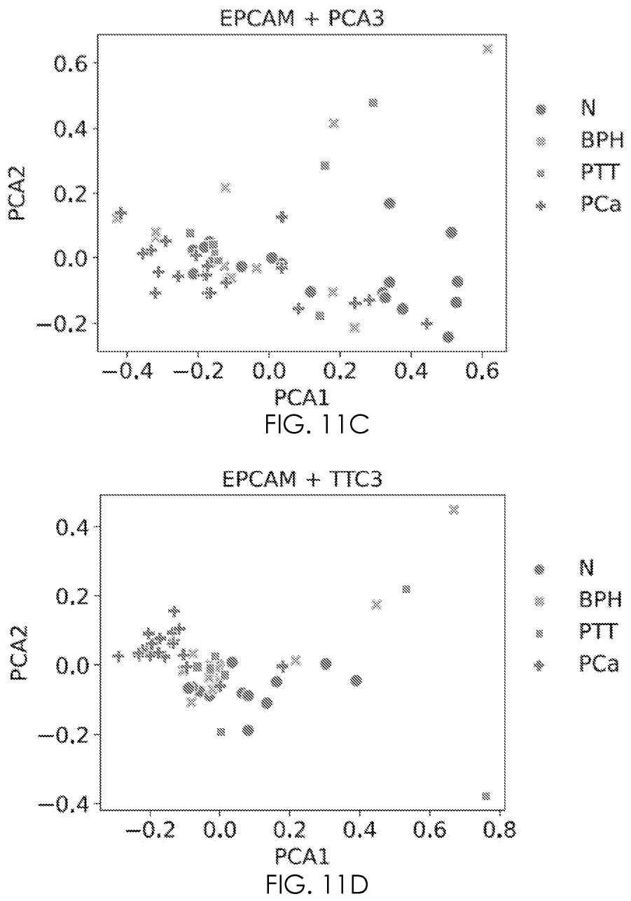

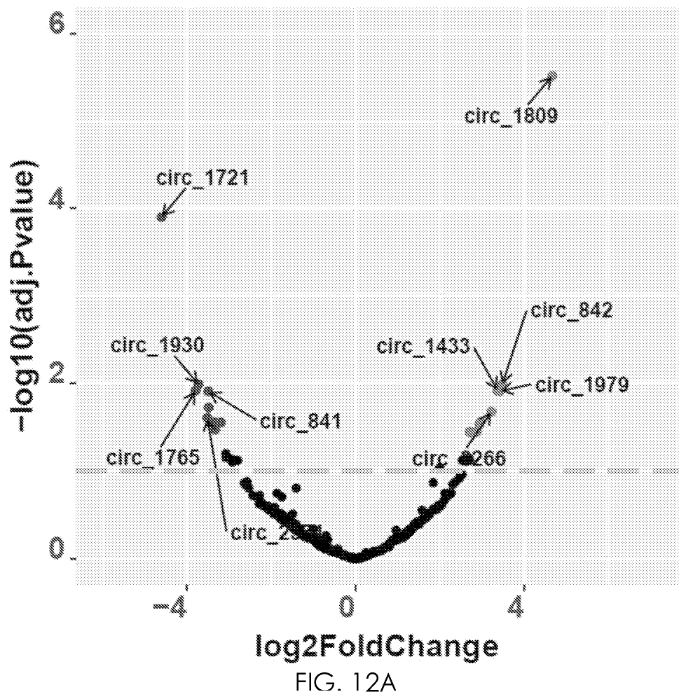

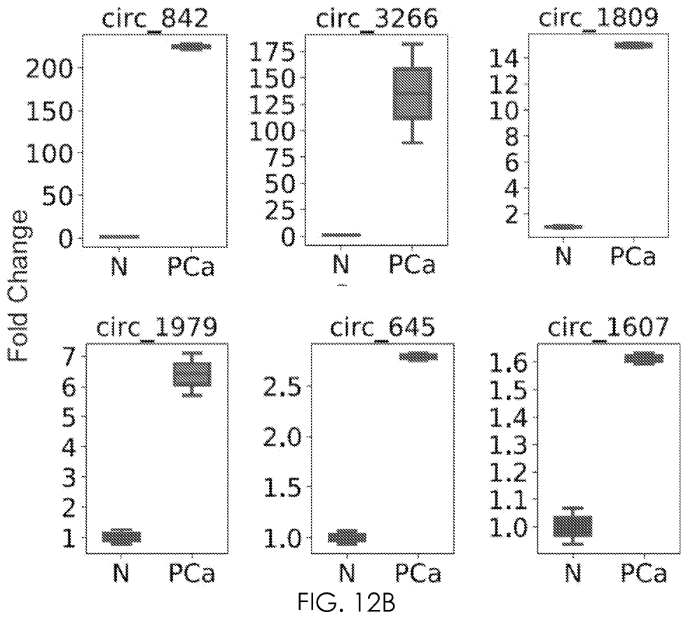

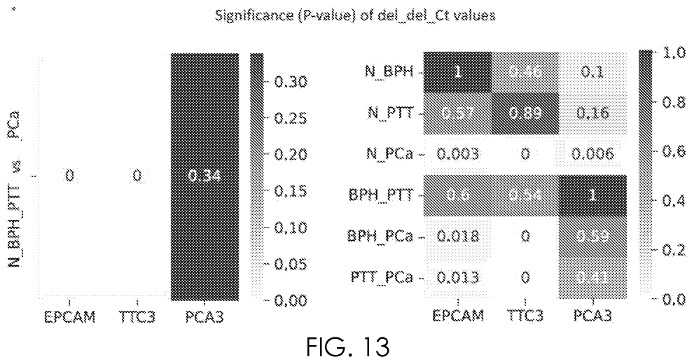

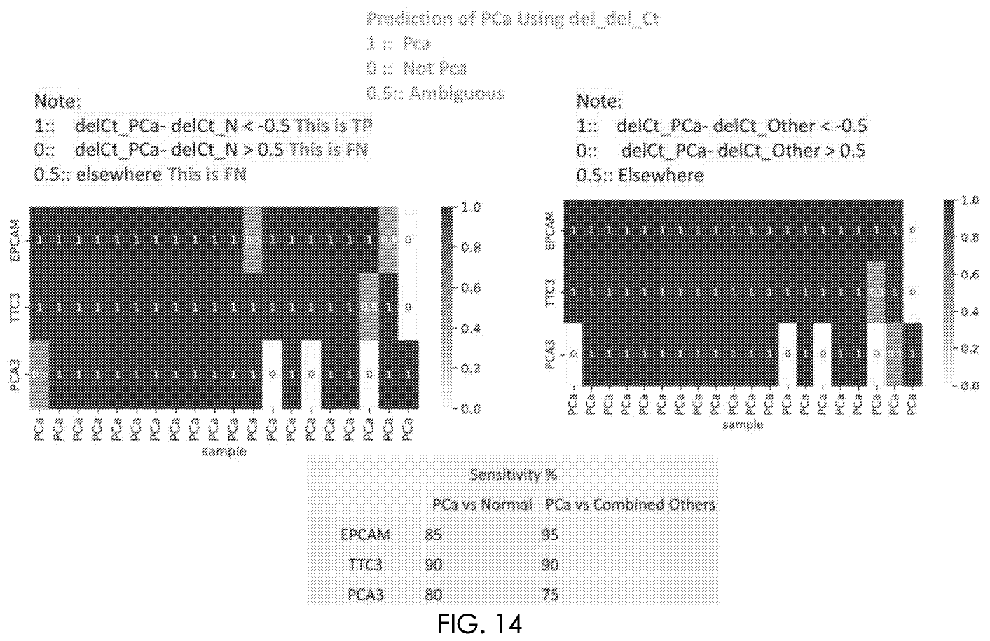

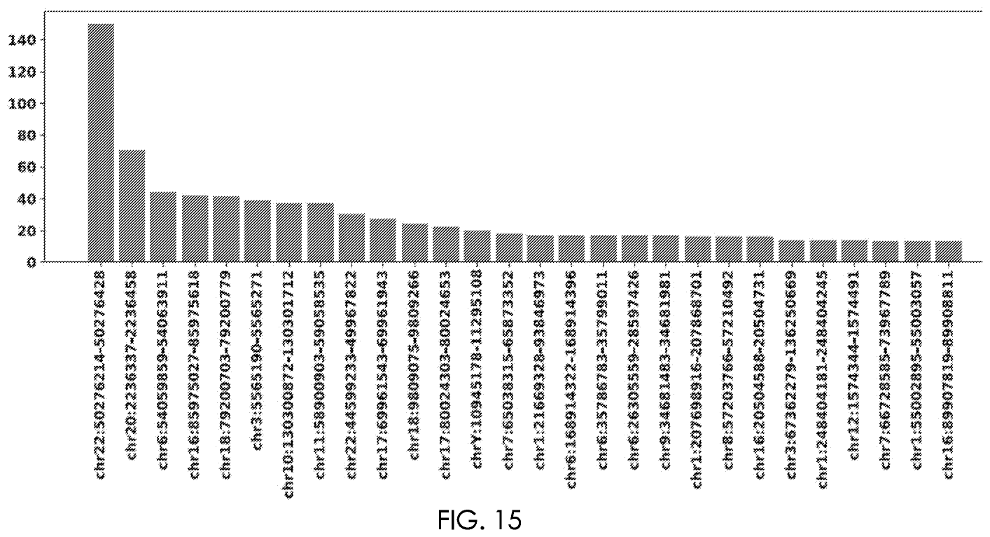

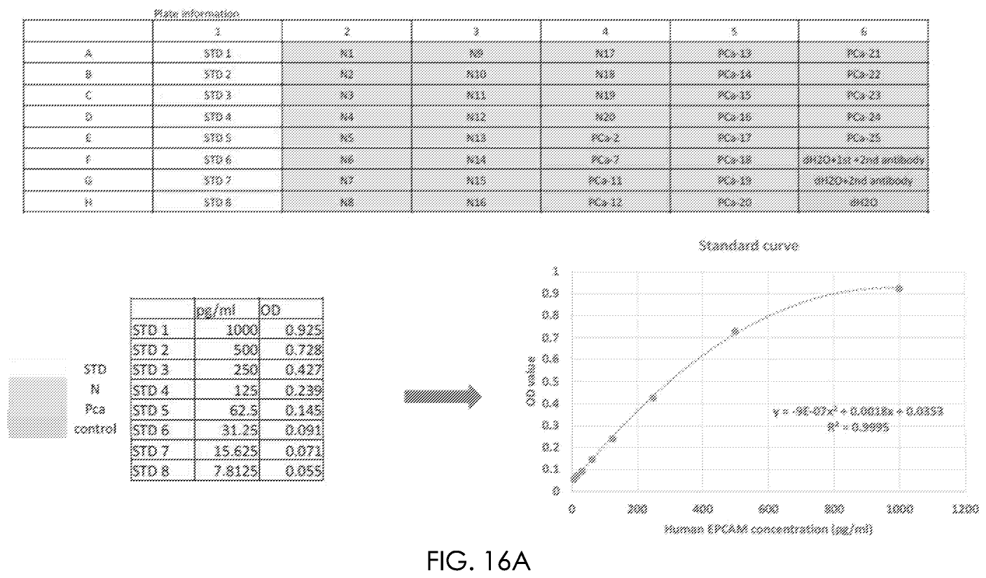

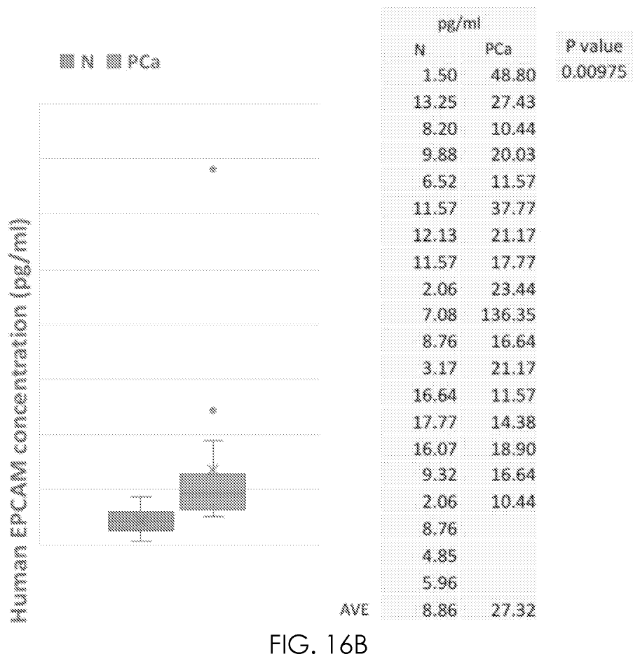

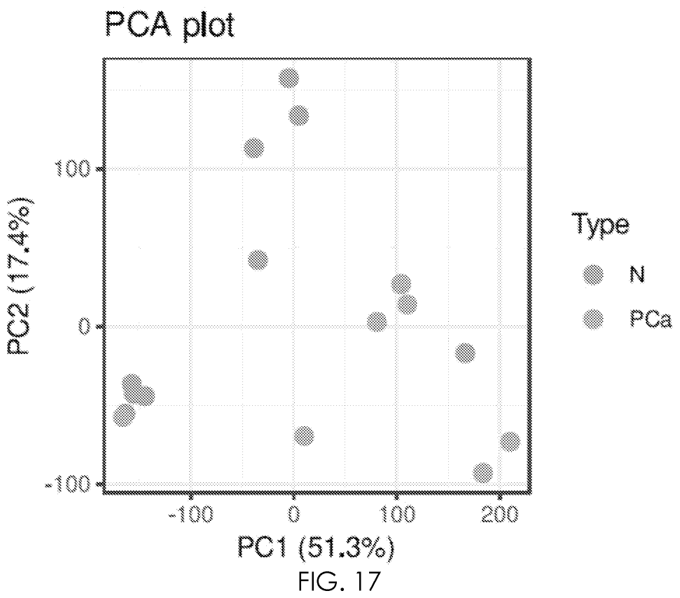

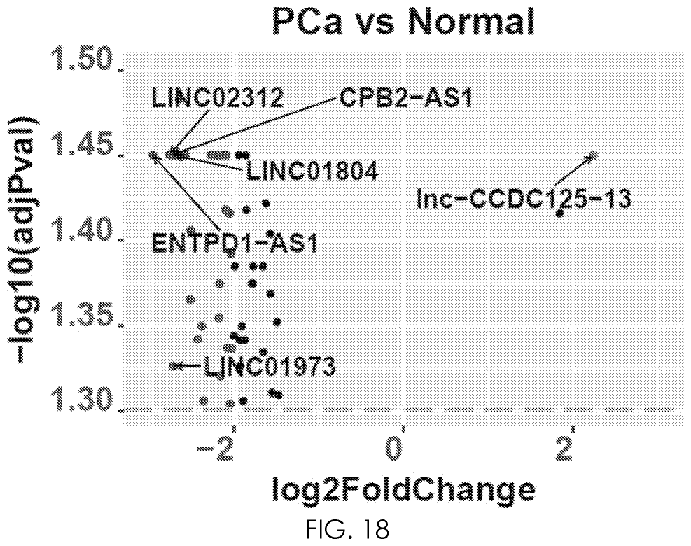

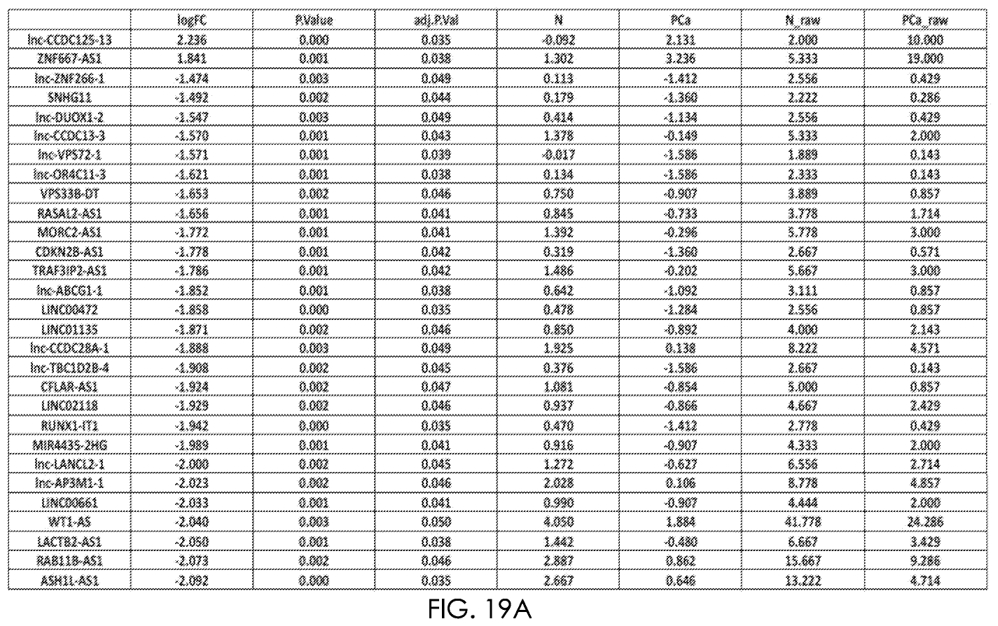

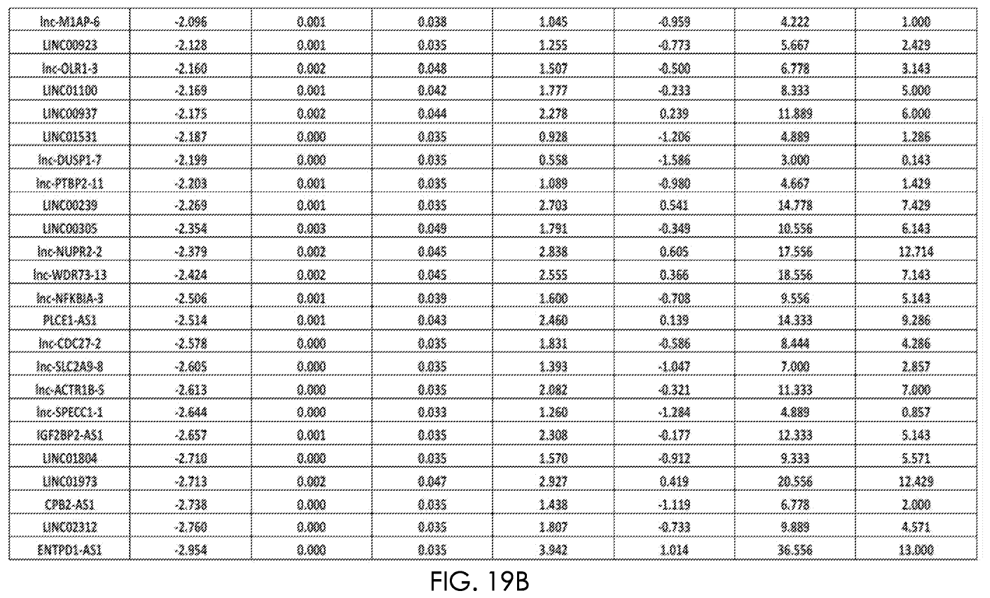

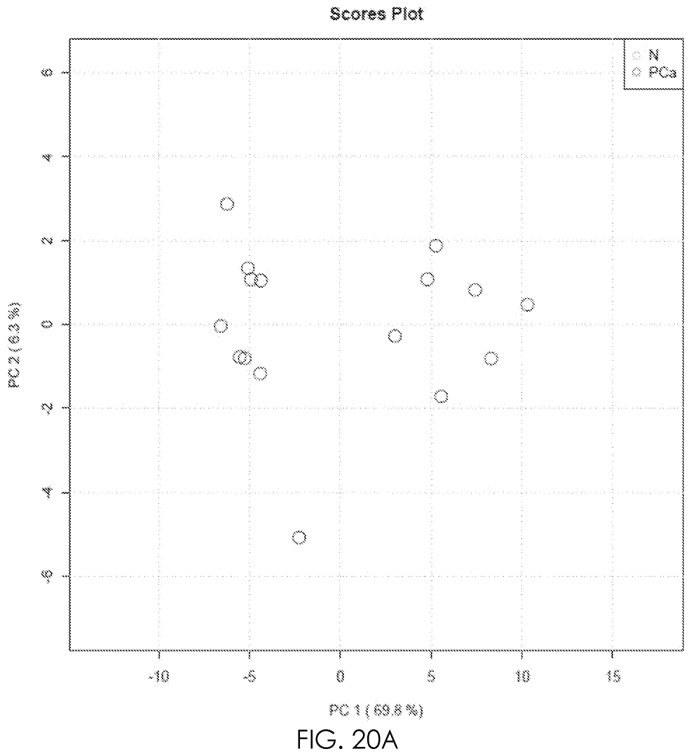

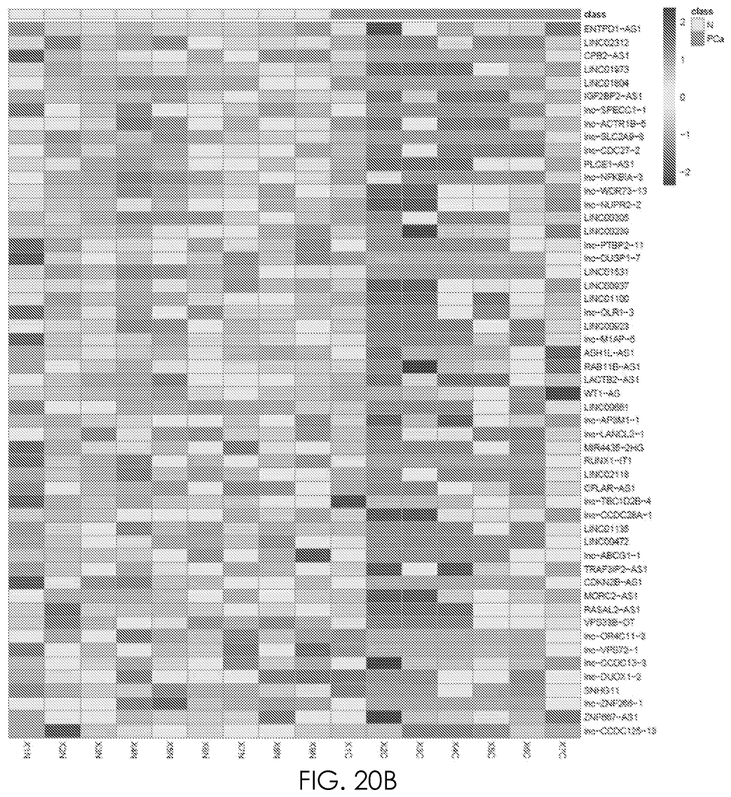

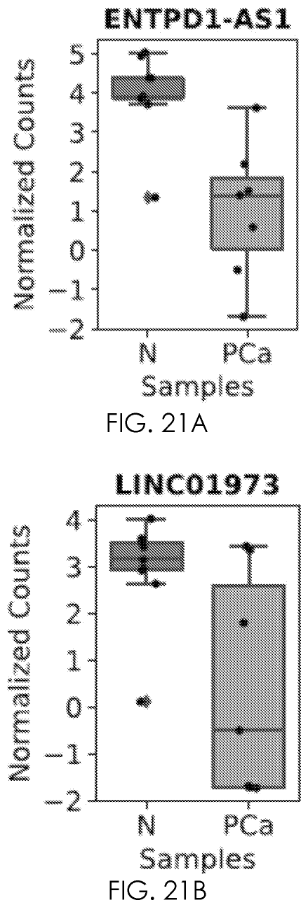

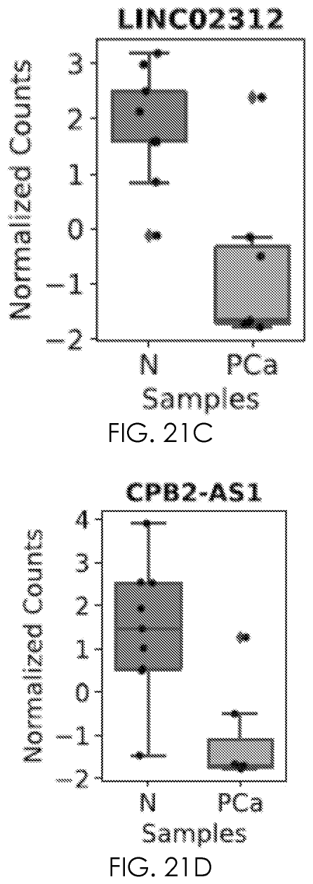

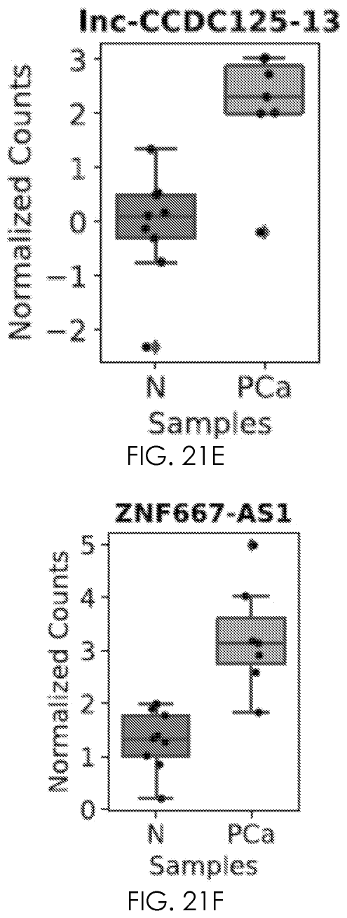

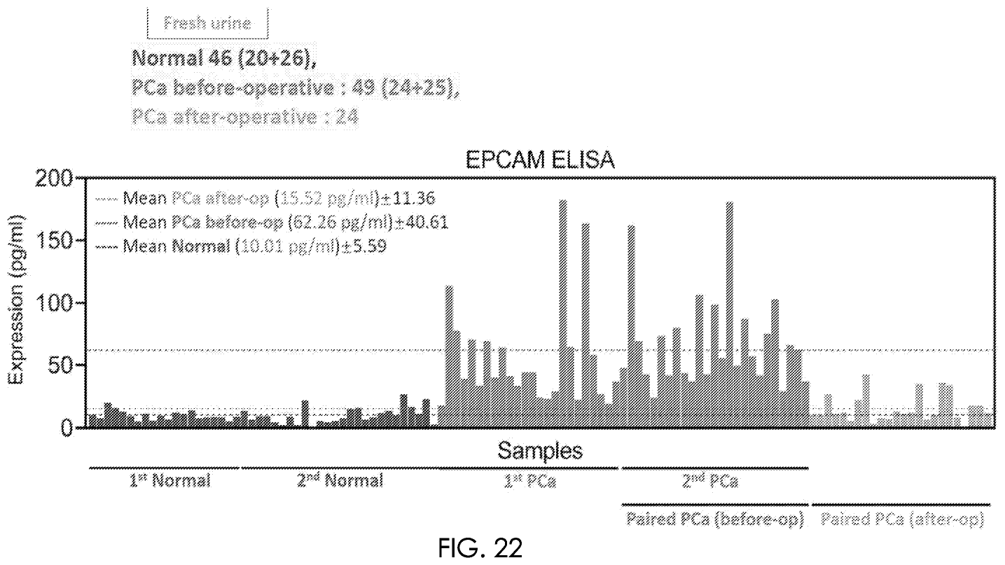

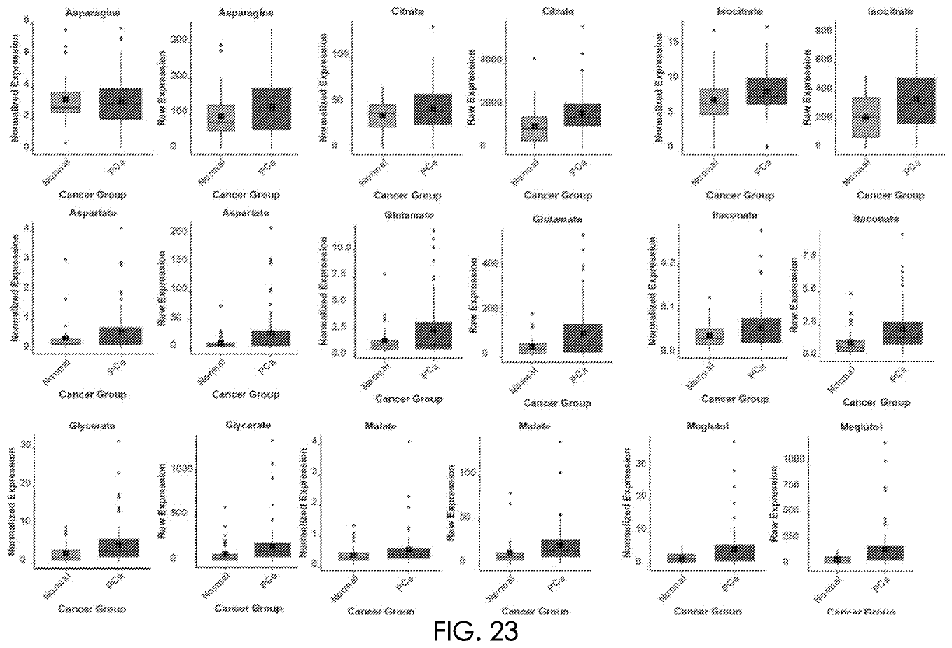

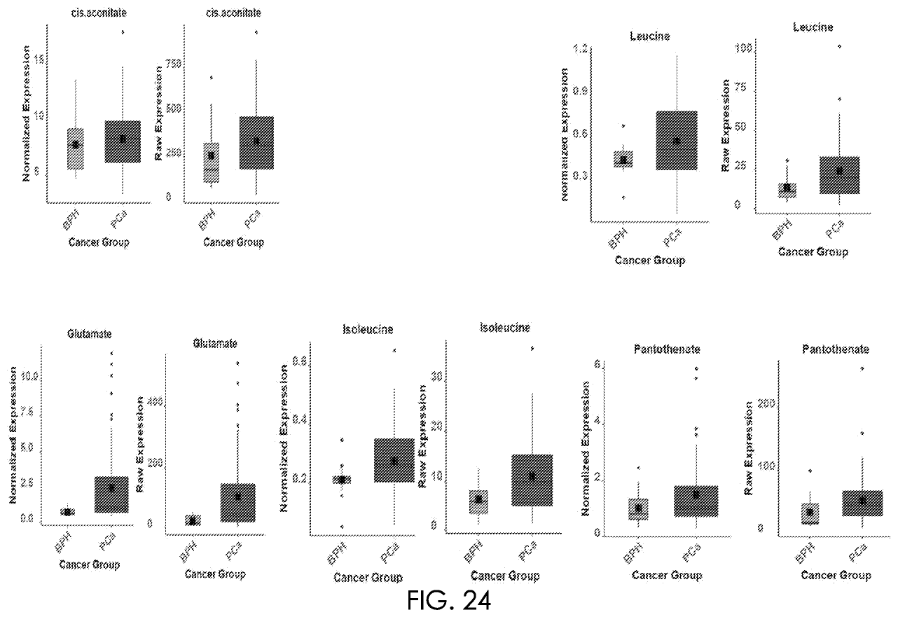

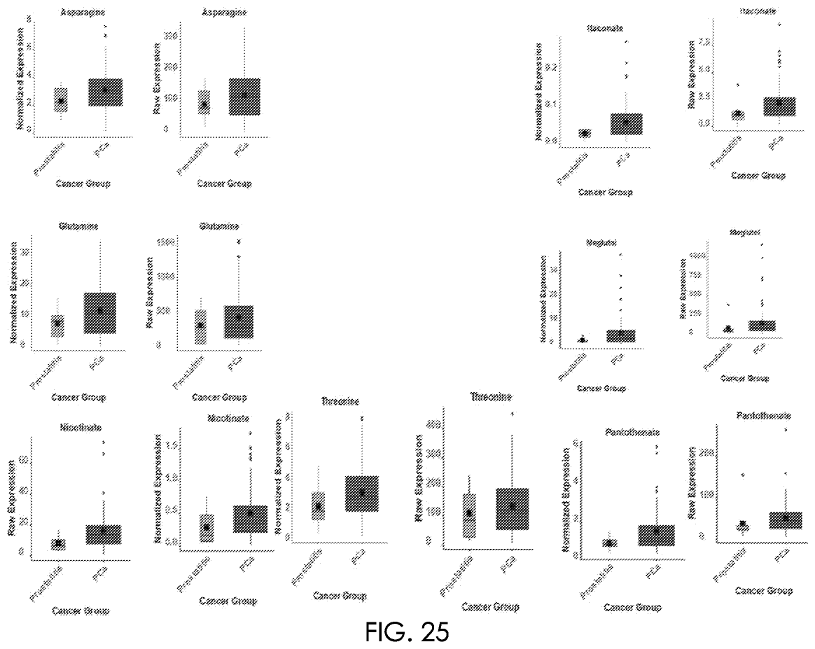

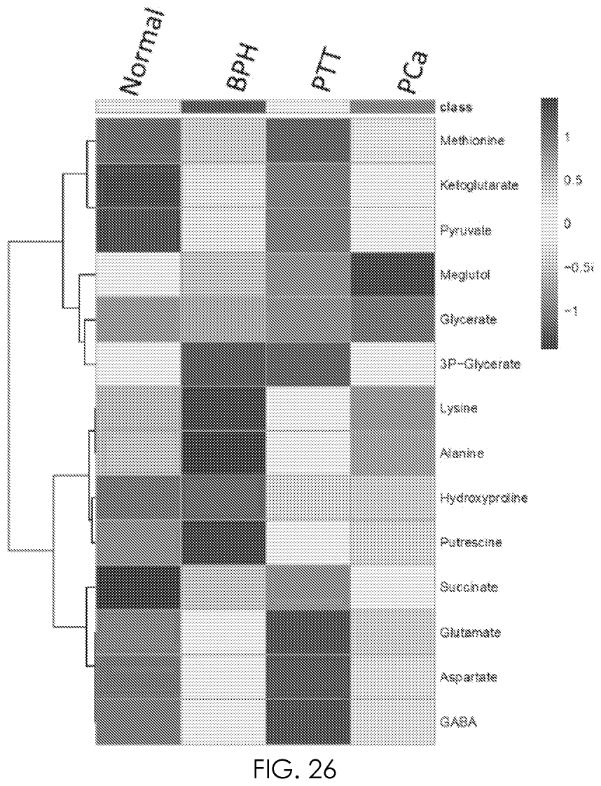

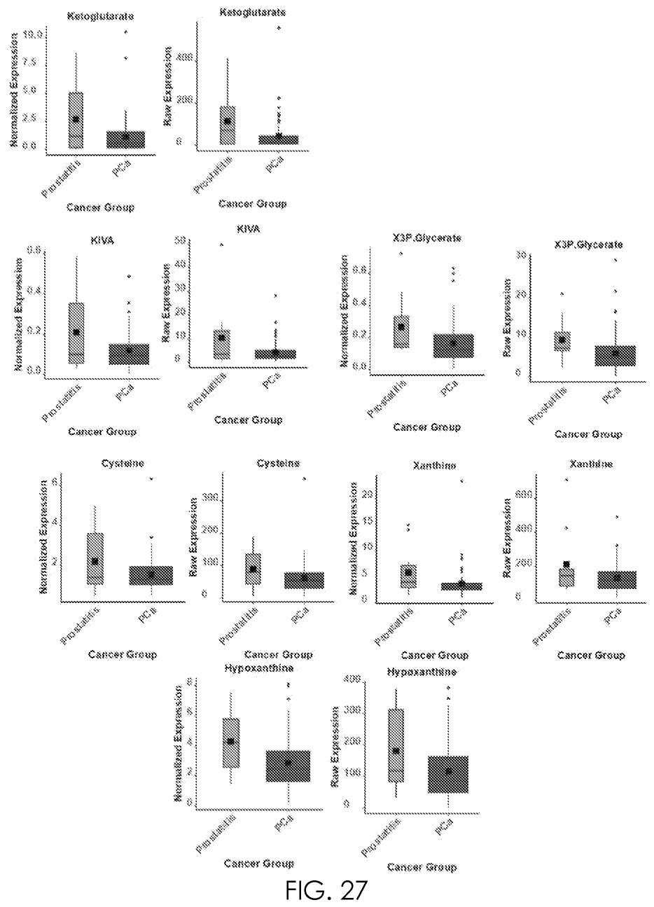

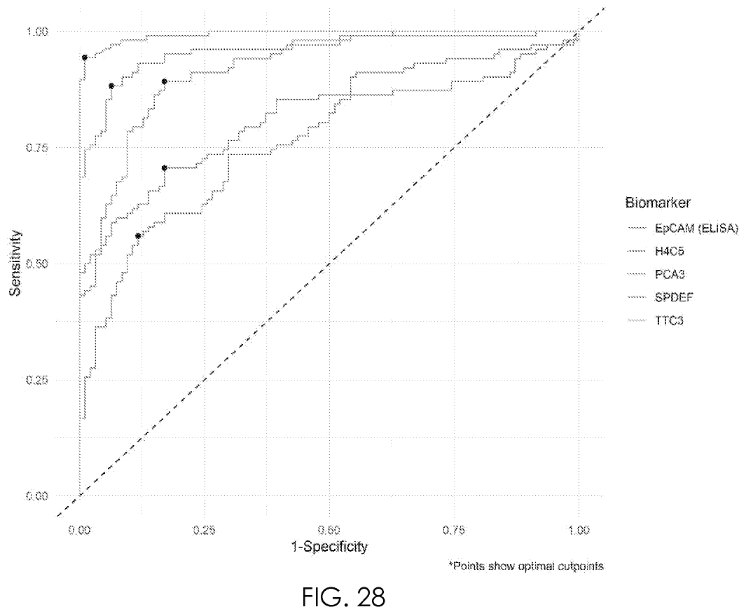

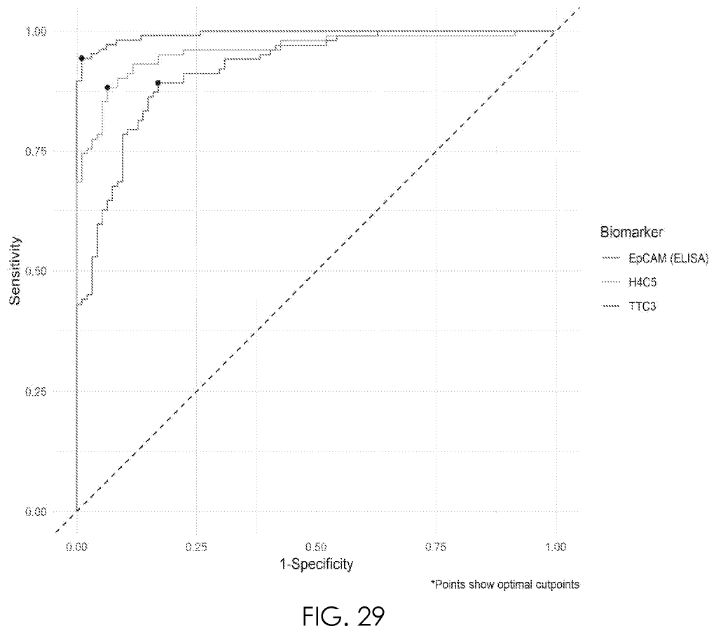

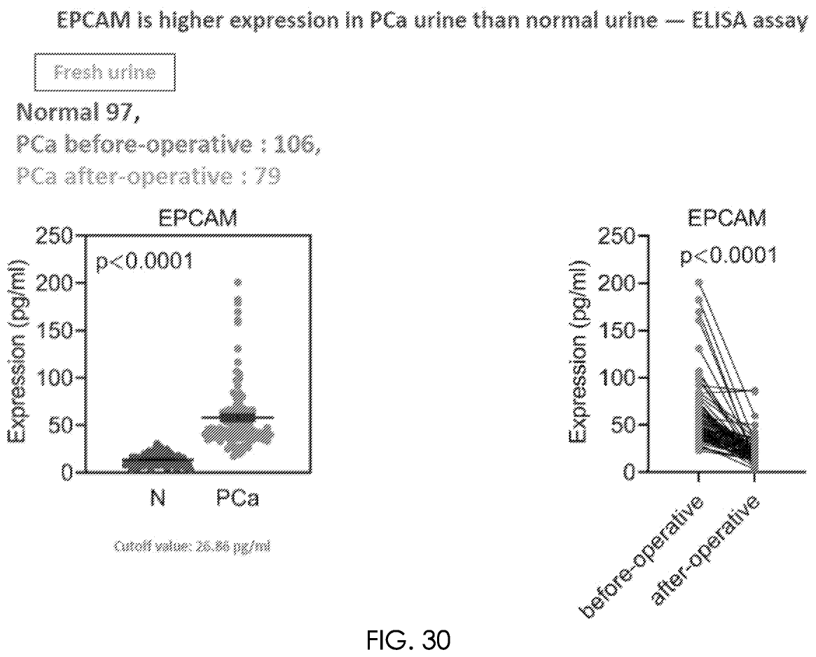

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

In a broad sense, the present invention contemplates the testing of one or more classes of biomarker molecules. The classes of molecules can be selected from polypeptide/protein, nucleic acid and poly amino acids, as well as metabolites. Nucleic acid molecules include deoxyribonucleic acid (DNA) including genomic DNA, plasmid DNA, complementary DNA (cDNA), cell-free (e.g., non-encapsulated) DNA (cfDNA) (also referred to as extracellular DNA (eccDNA), circulating tumor DNA (ctDNA), nucleosomal DNA, chromosomal DNA, mitochondrial DNA (miDNA), an artificial nucleic acid analog, recombinant nucleic acid, plasmids, viral vectors, and chromatin. In particular embodiments, the patient sample comprises eccDNA/cfDNA.

Nucleic acid molecules can also include ribonucleic acid (RNA) including coding and non-coding transcripts, messenger RNA (mRNA), transfer RNA (tRNA), micro RNA (mitoRNA), ribosomal RNA (rRNA), circulating RNA (cRNA), alternatively spliced mRNAs, long non-coding RNA (lncRNA), small nuclear RNAs (snRNAs), antisense RNA, short hairpin RNA (shRNA), or small interfering RNA (siRNA). In particular embodiments, the patient sample comprises mRNA, circRNA, and/or lncRNA.

A nucleic acid molecule or fragment thereof may comprise a single strand or can be double-stranded. A sample may comprise one or more types of nucleic acid molecules or fragments thereof.

A nucleic acid molecule or fragment thereof may comprise any number of nucleotides. For example, a single-stranded nucleic acid molecule or fragment thereof may comprise at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 220, at least 240, at least 260, at least 280, at least 300, at least 350, at least 400, or more nucleotides, In the instance of a double-stranded nucleic acid molecule or fragment thereof, the nucleic acid molecule or fragment thereof may comprise at least 10, at least 20, at least 30, at least 40, at least 50, at least 60, at least 70, at least 80, at least 90, at least 100, at least 1 10, at least 120, at least 130, at least 140, at least 150, at least 160, at least 170, at least 180, at least 190, at least 200, at least 220, at least 240, at least 260, at least 280, at least 300, at least 350, at least 400, or more basepairs (bp), e.g. pairs of nucleotides. In some cases, a double-stranded nucleic acid molecule or fragment thereof may comprise between 100 and 200 bp, such as between 120 and 180 bp. For example, the sample may comprise a cfDNA molecule that comprises between 120 and 180 bp.

The classes of biomarker molecules can also include polyamino acids comprising poly amino acid, peptide, or proteins. As used herein the term polyamino acid refers to a polymer in which the monomers are amino acid residues which are joined together through amide bonds. When the amino acids are alpha-amino acids, either the L-optical isomer or the D-optical isomer can be used, the L-isomers being preferred. In one example, the analyte is an autoantibody.

Further examples of the classes of molecules (or analytes) include metabolites such as sugars, lipids, amino acids, fatty acids, phenolic compounds, or alkaloids. In one embodiment, the analyte is a carbohydrate. In another embodiment, the analyte is a carbohydrate antigen. In a more specific embodiment, the carbohydrate antigen is attached to an O-glycan. In certain embodiments, the analyte is a mono- di-, tri- or tetra-saccharide.

In specific embodiments, the biomarker classes include RNA (mRNA, circRNA, lncRNA), eccDNA, proteins, metabolites and combinations of the foregoing.

A sample comprising one or more analytes/classes of biomarkers can be processed to provide or purify a particular analyte or a collection thereof. In one embodiment, a sample comprising one or more analytes can be processed to separate one type of analyte (e.g., protein or eccDNA/cfDNA) from other types of analytes (e.g., mRNA, circRNA, lncRNA). In another embodiment, the sample is separated into aliquots for analysis of a different analyte in each aliquot from the sample. In yet another embodiment, a sample comprising one or more nucleic acid molecules or fragments thereof of different sizes (e.g., lengths) can be processed to remove higher molecular weight and/or longer nucleic acid molecules or fragments thereof or lower molecular weight and/or shorter nucleic acid molecules or fragments thereof.

Sample processing may comprise, for example, one or more processes such as centrifugation, filtration, selective precipitation, tagging, barcoding, and partitioning. For example, cellular DNA can be separated from cfDNA by a selective polyethylene glycol and bead-based precipitation process such as a centrifugation or filtration process.

As described further herein, assays useful for detecting biomarkers of the present invention include, but are not limited to, whole-genome sequencing (WGS), whole-genome bisulfite sequencing (WGSB), small-RNA sequencing, quantitative immunoassay, enzyme-linked immunosorbent assay-(ELISA), proximity extension assay (PEA), protein microarray, mass spectrometry, low-coverage Whole-Genome Sequencing (lcWGS), cf-Protein Immuno-Quant ELISAs, SIMOA; and cf-mi RNA sequencing, and cell type or cell phenotype mixture proportions derived from any of the above assays.

Analysis of biomarker detection can be performed by a classifier trained and constructed according to one or more of, but not limited to: linear discriminant analysis (LDA); partial least squares (PLS); random forest; principal component analysis (PCA); k-nearest neighbor (KNN); support vector machine (SVM) with radial basis function kernel (SVMRadial); SVM with linear basis function kernel (SVMLinear); SVM with polynomial basis function kernel (SVNMPoly), decision trees, multilayer perceptron, mixture of experts, sparse factor analysis, hierarchical decomposition and combinations of linear algebra routines and statistics.

### I. Definitions

As used herein, a “subject”, “patient” or “individual” is a human. A subject can be one who has been previously diagnosed with or identified as suffering from or having a condition, disease, or disorder in need of treatment (e.g., prostate cancer) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a disease, a subject that has a disease, a subject diagnosed with a disease, a subject that has been treated for a disease, a subject that is being treated for a disease, and a subject that is at risk of developing a disease.

In some embodiments, the subject is selected from the group consisting of a subject suspected of having prostate cancer, a subject that has prostate cancer, a subject diagnosed with prostate cancer, a subject that has non-aggressive prostate cancer, a subject suspected of having aggressive prostate cancer, a subject that has been treated for prostate cancer, a subject having benign prostate hyperplasia, a subject having prostatitis, a subject that is being treated for prostate cancer, and a subject that is at risk of developing prostate cancer.

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made. In certain embodiments, a subject can be at risk of developing aggressive prostate cancer.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a serum sample. In particular embodiments, the sample is a urine sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples. In particular embodiments, body fluid means urine.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{1M}) samples can be stored and mailed, and an assay can be performed remotely.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function s in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “peptide” as used herein refers to any compound containing at least two amino acid residues joined by an amide bond formed from the carboxyl group of one amino acid residue and the amino group of the adjacent amino acid residue. In some embodiments, peptide refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In some embodiments, the peptide ranges in length from 2 to about 12 residues, or 2 to about 20 residues, or 2 to about 30 residues, or 2 to about 40 residues, or 2 to about 50 residues, or 2 to about 60 residues, or 2 to about 70 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chern. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. 11th American Peptide Symposium, ESCOM Science Publishers, The Netherlands, and the like)).

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide,” A protein contains at least one polypeptide. Short polypeptides, e.g., containing less than 20-30 residues, are sometimes referred to as “peptides.”

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy-chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the biomarkers are detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

The term “statistically significant” or “significantly” refers to statistical evidence that there is a difference. It is defined as the probability of making a decision to reject the null hypothesis when the null hypothesis is actually true. The decision is often made using the p-value.

As used herein, the term “sensitivity” refers to the ability of a method to detect or identify the presence of a disease in a subject. For example, when used in reference to any of the variety of methods described herein that can detect the presence of cancer in a subject (e.g., aggressive prostate cancer), a high sensitivity means that the method correctly identifies the presence of aggressive prostate cancer in the subject a large percentage of the time. For example, a method described herein that correctly detects aggressive prostate cancer in a subject 95% of the time the method is performed is said to have a sensitivity of 95%. In particular embodiments, a method described herein that can detect aggressive prostate cancer in a subject (or distinguish between aggressive and non-aggressive prostate cancer) provides a sensitivity of at least 70% (e.g., about 70%, about 72%, about 75%, about 80%, about 85%, about 90%, about 91%, about 92%, about 93%, about 94%, about 95%, about 96%, about 97%, about 98%, about 99%, about 99.5%, or about 100%). In certain embodiments, methods provided herein that include detecting the presence of one or more members of two or more classes of biomarkers (e.g., nucleic acid biomarkers and/or protein biomarkers) provide a higher sensitivity than methods that include detecting the presence of one or more members of only one class of biomarkers.

As used herein, the term “specificity” refers to the ability of a method to detect the presence of a disease in a subject (e.g., the specificity of a method can be described as the ability of the method to identify the true positive over true negative rate in a subject and/or to distinguish a truly occurring sequence variant from a sequencing artifact or other closely related sequences). For example, when used in reference to any of the variety of methods described herein that can detect the presence of cancer (e.g., aggressive prostate cancer) in a subject, a high specificity means that the method correctly identifies the absence of cancer in the subject a large percentage of the time (e.g., the method does not incorrectly identify the presence of cancer in the subject a large percentage of the time). In some embodiments, a method described herein that can detect the absence of cancer (normal, BPH or otherwise non-aggressive cancer) in a subject provides a specificity of at least 80% (e.g., at least 80%, 85%, 90%, 91%, 92%, 93%, 94%, 95%, 96%, 97%, 98%, 99%, 99.5%, or higher). A method having high specificity results in minimal or no false positive results (e.g., as compared to other methods). False positive results can arise from any source. In some embodiments, methods provided herein that include detecting the presence of one or more members of two or more classes of biomarkers (e.g., nucleic acid biomarkers and/or protein biomarkers) provide a higher specificity than methods that include detecting the presence of one or more members of only one class of biomarkers.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous when mere detection indicates the diagnosis. The term is also used synonymously with the term “measuring.” The terms “marker” or “biomarker” are used interchangeably herein, and in the context of the present invention refer to a protein or peptide (for example, protein or peptide associated with prostate cancer or prostate cancer as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof. In particular embodiments, a marker or biomarkers comprises an RNA (e.g., circulating RNA (circRNA), lncRNA, mRNA), a DNA (e.g., extracellular DNA (eccDNA) (also known as cell-free DNA or cfDNA), a peptide/protein, and/or a metabolite. In certain embodiments, the marker or biomarkers are measured in urine.

A “test amount” of a marker refers to an amount of a marker present in a sample being tested. A test amount can be either in absolute amount (e.g., g/mL) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., g/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed, A control amount can be either in absolute amount (e.g., pg/ml) or a relative amount (e.g., relative intensity of signals).

The term “differentially present” or “change in level” refers to differences in the quantity and/or the frequency of a marker present in a sample taken from patients having a specific disease or disorder as compared to a control subject. For example, a marker can be present at an elevated level or at a decreased level in samples of patients with the disease or disorder compared to a control value (e.g., determined from samples of control subjects). Alternatively, a marker can be detected at a higher frequency or at a lower frequency in samples of patients compared to samples of control subjects. A marker can be differentially present in terms of quantity, frequency or both as well as a ratio of differences between two or more specific modified amino acid residues and/or the protein itself. In one embodiment, an increase in the ratio of modified to unmodified proteins and peptides described herein is diagnostic of any one or more of the diseases described herein. In particular embodiments, a marker can be differentially present in patients having aggressive prostate cancer as compared to a control subject including patients having non-aggressive prostate cancer or no cancer. Differentially present can refer to PCa versus other conditions including normal, BPH and/or PTT.

A marker, compound, composition or substance is differentially present in a sample if the amount of the marker, compound, composition or substance in the sample (a patient having aggressive prostate cancer) is statistically significantly different from the amount of the marker, compound, composition or substance in another sample (a patient having non-aggressive cancer or no cancer), or from a control value (e.g., an index or value representative of non-aggressive cancer or no cancer). For example, a marker is differentially present if it is present at least about 50%, at least about 60%, at least about 70%, at least about 80%, at least about 90%, at least about 100%, at least about 110%, at least about 120%, at least about 130%, at least about 150%, at least about 180%, at least about 200%, at least about 300%, at least about 500%, at least about 700%, at least about 900%, or at least about 1000% greater or less than it is presence in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or disorder, is statistically significantly higher or lower than in the control samples or control values obtained from controls such as a subject having non-aggressive prostate cancer, benign lesions and the like, or otherwise healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples (e.g., a patient having aggressive prostate cancer) than the other set of samples (e.g., a patient having non-aggressive prostate cancer or no cancer). These exemplary values notwithstanding, it is expected that a skilled practitioner can determine cut-off points, etc., that represent a statistically significant difference to determine whether the marker is differentially present.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include ³²P, ³¹S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

### II. Detection/Measurement of Nucleic Acid Markers

Nucleic acids may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing.

Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR).

Sequencing reads can be obtained from various sources including, for example, whole genome sequencing, whole exome-sequencing, targeted sequencing, next-generation sequencing, pyrosequencing, sequencing-by-synthesis, ion semiconductor sequencing, tag-based next generation sequencing semiconductor sequencing, single-molecule sequencing, nanopore sequencing, sequencing-by-ligation, sequencing-by-hybridization, Digital Gene Expression (DGE), massively parallel sequencing, Clonal Single Molecule Array (Solexa/Illumina), sequencing using PacBio, and Sequencing by Oligonucleotide Ligation and Detection (SOLiD).

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (i.e., a UMI). In specific embodiments, a barcode is non-unique, and barcode sequences can be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule).

Sequencing reads may be processed using methods such as de-multiplexing, de-deduplication (e.g., using unique molecular identifiers, UMIs), adapter-trimming, quality filtering, GC correction, amplification bias correction, correction of batch effects, depth normalization, removal of sex chromosomes, and removal of poor-quality genomic bins.)

In various embodiments, sequencing reads may be aligned to a reference nucleic acid sequence. In one example, the reference nucleic acid sequence is a human reference genome. As examples, the human reference genomne can be hg19, hg38, GrCH38, GrCH37, NA12878, or GM12878.

### III. Detection/Measurement of Protein Markers

In specific embodiments, the proteins of the present invention can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. In other embodiments, the biospecific capture reagents bind the specific biomarker and not similar forms thereof.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the protein biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarker; and detecting binding of the antibodies, or antigen binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

For example, the level of a biomarker in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the protein. The detection can be performed using a second antibody to bind to the capture antibody complexed with its target biomarker. A target biomarker can be an entire protein, or a variant or modified form thereof. Kits for the detection of proteins as described herein can include pre-coated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

The present disclosure also provides methods for detecting protein in a sample obtained from a subject, wherein the levels of expression of the proteins in a biological sample are determined simultaneously. For example, in one embodiment, methods are provided that comprise: (a) contacting a biological sample obtained from the subject with a plurality of binding agents that each selectively bind to one or more biomarker proteins for a period of time sufficient to form binding agent-biomarker complexes; and (b) detecting binding of the binding agents to the one or more biomarker proteins. In further embodiments, detection thereby determines the levels of expression of the biomarkers in the biological sample; and the method can further comprise (c) comparing the levels of expression of the one or more biomarker proteins in the biological sample with predetermined threshold values, wherein levels of expression of at least one of the biomarker proteins above or below the predetermined threshold values indicates, for example, the subject has prostate cancer, the severity of prostate cancer, and/or is/will be responsive to prostate cancer therapy. Such embodiments can assist in identifying whether a subject has PCa versus normal, BPH and/or PTT. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above-described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, lectins, peptides, aptamer, etc., combinations thereof) to form a biomarker:capture agent complex. The complexes can then be detected and/or quantified. A subject can then be identified as having aggressive prostate cancer based on a comparison of the detected/quantified/measured levels of biomarkers to one or more reference controls as described herein.

In one method, a first, or capture, binding agent, such as an antibody that specifically binds the protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In a more specific embodiment, a biotinylated lectin that specifically binds a biomarker can be added to a patient sample and a streptavidin labeled fluorescent marker that binds the biotinylated lectin bound to the biomarker is then added, and the biomarker is detected.

In another embodiment, the assay is a competitive binding assay, wherein labeled protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

In several embodiments, the biomarkers of the present invention may be detected by means of an electrochemiluminescent assay developed by Meso Scale Discovery (Gaithersburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The proteins of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In particular embodiments, the protein biomarker proteins of the present invention can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of blood serum or plasma are dropped on the chip array. Protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### IV. Detection/Measurement of Metabolites

Metabolites useful in the present invention include, but are not limited to, asparagine, aspartate, glycerate, citrate, isocitrate, glutamate, itaconate, malate, meglutol, cis-aconitate, isoleucine, leucine, pantothenate, glutamine, nicotinate, and threonine. Compositions and methods for detecting/measuring metabolites are known in the art. See, e.g., Metabolon, Inc. (Morrisville, NC) (e.g., U.S. Pat. No. 10,890,592; 11,181,530; 11,061,005; 10,965,183; 10,573,406; and 10,267,777); Abcam plc (Cambridge, UK) (e.g., Asparagine Assay Kit (Fluorometric), Glutamine Assay Kit (Colorimetric), Aspartate Assay Kit, and Citrate Assay Kit); Promega Corporation (Madison, WI) (e.g., Glutamate-Glo™ Assay, and Glutamine/Glutamate-Glo® Assay); and Sigma-Aldrich, Inc. (St. Louis, MO) (e.g., Glutatmate Assay Kit, Citrate Assay Kit, Isocitrate Assay Kit, and Malate Assay Kit) In other embodiments, the metabolite biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, metabolites are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. Nos. 6,225,047 and 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### V. Point-of-Care Assays for Detecting Target Proteins/Nucleic Acids

The types of assays described above are amenable to developing point-of-care (POC) devices, in which systems can be self-contained so that output is readable by the user. This characteristic is especially useful when collection of a sample to be tested does not require medical intervention (e.g., urine, saliva, or sputum). One device that enables this is the lateral-flow device (LFD). These devices use a multi-layered construction containing both absorbent and non-absorbent components to form a solid-phase. The capture and/or recognition reagents (antigen or antibody) are pre-applied to specific areas within the assembled apparatus and the analyte is allowed to flow through the system to come into contact with reagents. Often, for the purpose of self-containment, the reagent components are added in a dried state so that fluid from the sample re-hydrates and activates them. Conventional ELISA techniques can then be used to detect the analyte in the antigen-antibody complex. In some embodiments, the system can be designed to provide a colorimetric reading for visual estimation of a binary response (‘yes’ or ‘no’), or it can be configured to be quantitative.

Although many of the embodiments contemplated herein with respect to lateral flow devices are described in terms of detecting proteins (e.g., EPCAM), lateral flow can be used to detect nucleic acids including, but not limited to, H4C5 and TTC3 (as well as other nucleic acid biomarkers described herein including mRNA, circRNA, eccDNA and lncRNA). See U.S. Pat. No. 9,121,849 (Rapid Pathogen Screening, Inc.); U.S. Patent Application Publication No. 20090305290 (Rapid Pathogen Screening, Inc.), and International Patent Application Publication No. WO2004/092342 (Applera Corporation). Metabolites or small molecular can also be detected using lateral flow technologies. See U.S. Pat. No. 8,399,261 (Inbios International, Inc.), (International Patent Application Publication No. 2017/075649) and Nuntawong et al., 76 J. NAT. MED. 521-45 (2022). Thus, it is contemplated herein that proteins, nucleic acids and small molecules (e.g., metabolites) can be detected via lateral flow. In particular embodiments, a lateral flow assay is used to detect EpCAM and qPCR is used to detect H4C5 and TTC3.

In certain embodiments, the presently disclosed methods can use a lateral flow device or dipstick assay comprising an immunochromatographic strip test that relies on a direct (double antibody sandwich) reaction. Without wishing to be bound to any one particular theory, this direct reaction scheme can be used when sampling for larger analytes that may have multiple antigenic sites. Different antibody combinations can be used, for example different antibodies can be included on the capture (detection) line, the control line, and included in the mobile phase of the assay, for example, as conjugated to gold particles, e.g., gold microparticles, gold nanoparticles, or fluorescent dyes.

The term “dipstick assay” as used herein means any assay using a dipstick in which sample solution is contacted with the dipstick to cause sample solution to move by capillary action to a capture zone of the dipstick thereby allowing a target antigen in the sample solution to be captured and detected at the capture zone. To test for the presence of analyte, the contact end of the dipstick is contacted with the test solution. If analyte is present in the test solution it travels to the capture zone of the dipstick by capillary action where it is captured by the capture antibody. The presence of analyte at the capture zone of the dipstick is detected by a further anti-analyte antibody (the detection antibody) labelled with, for example, colloidal gold.

These dipstick tests have several advantages. They are easy and cheap to perform, no specialist instruments are required, and the results are obtained rapidly and can be read visually. These tests are, therefore, particularly suited for use in a physician's office, at home, in remote areas, and in developing countries where specialist equipment may not be available. They can be used, for example, to detect PCa.

To perform a method of the first aspect of the invention, the targeting agent and labels may simply be added to the test solution and the test solution then contacted with the contact end of the chromatographic strip. Such methods are easier to perform than the method disclosed in WO 00/25135 in which two separate wicking steps are required. The results may, therefore, be obtained more rapidly, and yet the sensitivity of analyte detection is higher.

The term “chromatographic strip” is used herein to mean any porous strip of material capable of transporting a solution by capillary action. The chromatographic strip may be capable of bibulous or non-bibulous lateral flow, but preferably bibulous lateral flow. By the term “non-bibulous lateral flow” is meant liquid flow in which all of the dissolved or dispersed components of the liquid are carried at substantially equal rates and with relatively unimpaired flow laterally through the membrane as opposed to preferential retention of one or more components as would occur with “bibulous lateral flow.” Materials capable of bibulous lateral flow include paper, nitrocellulose, and nylon. A preferred example is nitrocellulose.

The labels may be bound to the targeting agent by pre-mixing the targeting agent with the labels before the targeting agent is added to (or otherwise contacted with) the test solution. However, in some circumstances, it is preferred that the targeting agent and labels are not pre-mixed because such pre-mixing can cause the targeting agent and labels to precipitate. Thus, the targeting agent and the labels may be added separately to (or contacted separately with) the test solution. The targeting agent and the labels can be added to (or contacted with) the test solution at substantially the same time, or in any order.

The test solution may be pre-incubated with the targeting agent and labels before the test solution is contacted with the contact end of the chromatographic strip to ensure complex formation. The optimal time of pre-incubation will depend on the ratio of the reagents and the flow rate of the chromatographic strip. In some cases, pre-incubation for too long can decrease the detection signal obtained, and even lead to false positive detection signals. Thus, it may be necessary to optimize the pre-incubation time for the particular conditions used.

It may be desired to pre-incubate the targeting agent with the test solution before binding the labels to the targeting agent so that the targeting agent can be allowed to bind to analyte in the test solution under optimum binding conditions.

As used herein the term “lateral flow” refers to liquid flow along the plane of a substrate or carrier, e.g., a lateral flow membrane. In general, lateral flow devices comprise a strip (or a plurality of strips in fluid communication) of material capable of transporting a solution by capillary action, i.e., a wicking or chromatographic action, wherein different areas or zones in the strip(s) contain assay reagents, which are either diffusively or non-diffusively bound to the substrate, that produce a detectable signal as the solution is transported to or migrates through such zones. Typically, such assays comprise an application zone adapted to receive a liquid sample, a reagent zone spaced laterally from and in fluid communication with the application zone, and a detection zone spaced laterally from and in fluid communication with the reagent zone. The reagent zone can comprise a compound that is mobile in the liquid and capable of interacting with an analyte in the sample, e.g., to form an analyte-reagent complex, and/or with a molecule bound in the detection zone. The detection zone may comprise a binding molecule that is immobilized on the strip and is capable of interacting with the analyte and/or the reagent and/or an analyte-reagent complex to produce a detectable signal. Such assays can be used to detect an analyte in a sample through direct (sandwich assay) or competitive binding. Examples of lateral flow devices are provided in U.S. Pat. No. 6,194,220 to Malick et al., U.S. Pat. No. 5,998,221 to Malick et al, U.S. Pat. No. 5,798,273 to Shuler et al; and U.S. Pat. No. RE38,430 to Rosenstein.

In some embodiments, the presently disclosed methods can be used with an assay comprising a sandwich lateral flow or dipstick assay. In a sandwich assay, a liquid sample that may or may not contain an analyte of interest is applied to the application zone and allowed to pass into the reagent zone by capillary action. The term “analyte” as used herein refers to a target proteins including, but not limited to EPCAM, H4C5 and/or TTC3. In certain embodiments the presence or absence of an analyte in a sample is determined qualitatively. In other embodiments, a quantitative determination of the amount or concentration of analyte in the sample is determined. In other embodiments, H4C5 and/or TTC3 nucleic acids such as RNA can be detected. In a specific embodiment, a target analyte is EPCAM protein. In some embodiments, a target analyte comprises H4C5 and/or TTC3 RNA. Target analytes can be protein, nucleic acid or metabolites of any of the biomarkers described herein.

The analyte, if present, interacts with a labeled reagent in the reagent zone to form an analyte-reagent complex and the analyte-reagent complex moves by capillary action to the detection zone. The analyte-reagent complex becomes trapped in the detection zone by interacting with a binding molecule specific for the analyte and/or reagent. Unbound sample can pass through the detection zone by capillary action to a control zone or an absorbent pad laterally juxtaposed and in fluid communication with the detection zone. The labeled reagent may then be detected in the detection zone by appropriate means.

Generally, and without limitation, lateral flow devices comprise a sample pad. A sample pad comprises a membrane surface, also referred to herein as a “sample application zone,” adapted to receive a liquid sample. A standard cellulose sample pad has been shown to facilitate absorption and flow of biological samples, including, but not limited to, urine. The sample pad comprises a portion of lateral flow device that is in direct contact with the liquid sample, that is, it receives the sample to be tested for the analyte of interest. The sample pad can be part of, or separate from, a lateral flow membrane. Accordingly, the liquid sample can migrate, through lateral or capillary flow, from sample pad toward a portion of the lateral flow membrane comprising a detection zone. The sample pad is in fluid communication with the lateral flow membrane comprising an analyte detection zone. This fluid communication can arise through or be an overlap, top-to-bottom, or an end-to-end fluid connection between the sample pad and a lateral flow membrane. In certain embodiments, the sample pad comprises a porous material, for example and not limited to, paper.

Typically, a sample pad is positioned adjacent to and in fluid communication with a conjugate pad. A conjugate pad comprises a labeled reagent having specificity for one or more analytes of interest. In some embodiments, the conjugate pad comprises a non-absorbent, synthetic material (e.g., polyester) to ensure release of its contents. A detection conjugate is dried into place on the conjugate pad and only released when the liquid sample is applied to the sample pad. Detection conjugate can be added to the pad by immersion or spraying.

In particular embodiments, the detection conjugate comprises an antibody that specifically binds EPCAM. In other embodiments, a detection conjugate comprises an antibody that specifically binds H4C5 and/or an antibody that specifically binds TTC3. In some embodiments, the antibody is a monoclonal antibody.

The antibody, e.g., a monoclonal antibody (MAb), can be conjugated to a fluorescent dye or gold particle, e.g., colloidal gold, including gold microspheres or gold nanoparticles, such as gold nanoparticles of about 40 nm. For example, it is possible to biotinylate the conjugated MAb to take advantage of the strong affinity that biotin has for streptavidin, using Streptavidin-coated microspheres. Alternatives include protein A-coated microspheres that bind to Fc region of IgGs.

In certain embodiments, the conjugate pad is adjacent to and in fluid communication with a lateral flow membrane. Capillary action draws a fluid mixture up the sample pad, through the conjugate pad where an antibody-antigen complex is formed, and into the lateral flow membrane. Lateral flow is a function of the properties of the lateral flow membrane. The lateral flow membrane typically is extremely thin and is hydrophilic enough to be wetted, thereby permitting unimpeded lateral flow and mixture of reactants and analytes at essentially the same rates.

Lateral flow membranes can comprise any substrate capable of providing liquid flow including, but not limited to, substrates, such as nitrocellulose, nitrocellulose blends with polyester or cellulose, untreated paper, porous paper, rayon, glass fiber, acrylonitrile copolymer, plastic, glass, or nylon. Lateral flow membranes can be porous. Typically, the pores of a lateral flow membrane are of sufficient size such that particles, e.g., microparticles comprising a reagent capable of forming a complex with an analyte, flow through the entirety of the membrane. Lateral flow membranes, in general, can have a pore size ranging from about 3 μm to about 100 μm, and, in some embodiments, have a pore size ranging from about m to about 50 μm. Pore size affects capillary flow rate and the overall performance of the device.

There are multiple benefits to using nitrocellulose for the primary membrane: low cost, capillary flow, high affinity for protein biding, and ease of handlisssssssng. Nitrocellulose has high protein binding. Another alternative is cellulose acetate, which has low protein binding. Size dictating surface area dictates membrane capacity (the volume of sample that can pass through the membrane per unit time=length×width×thickness×porosity. Because these variables control the rate at which lateral flow occurs, they can impact sensitivity and specificity of the assay. The flow rate also varies with sample viscosity. Several different sizes and polymers are available for use as microspheres, which migrate down the membrane with introduction of the fluidic sample. The optimal flow rate generally is achieved using spheres that are 1/10 the pore size of the membrane or smaller.

One skilled in the art will be aware of other materials that allow liquid flow. Lateral flow membranes, in some embodiments, can comprise one or more substrates in fluid communication. For example, a conjugate pad can be present on the same substrate or may be present on separate substrates (i.e., pads) within or in fluid communication with lateral flow membranes. In some embodiments, the nitrocellulose membrane can comprise a very thin Mylar sheet coated with a nitrocellulose layer.

Lateral flow membranes can further comprise at least one indicator zone or detection zone. The terms “indicator zone” and “detection zone” are used interchangeably herein and mean the portion of the carrier or porous membrane comprising an immobilized binding reagent. As used herein, the term “binding reagent” means any molecule or a molecule bound to a particle, wherein the molecule recognizes or binds the analyte in question. The binding reagent is capable of forming a binding complex with the analyte-labeled reagent complex. The binding reagent is immobilized in the detection zone and is not affected by the lateral flow of the liquid sample due to the immobilization on the membrane. Once the binding reagent binds the analyte-labeled reagent complex it prevents the analyte-labeled reagent complex from continuing with the flow of the liquid sample. In some embodiments, the binding reagent comprises an antibody that specifically binds EPCAM and an antibody that specifically binds H4C5. In other embodiments, the binding reagent further comprises an antibody that binds TTC3.

Accordingly, during the actual reaction between the analyte and the reagent, the first member binds in the indicator zone to the second member and the resulting bound complex is detected with specific antibodies. Detection may use any of a variety of labels and/or markers, e.g., enzymes (alkaline phosphatase or horseradish peroxidase with appropriate substrates), radioisotopes, liposomes or latex beads impregnated with fluorescent tags, polymer dyes or colored particles, and the like. Thus, the result can be interpreted by any direct or indirect reaction. Colloidal gold particles, which impart a purple or red coloration, are most commonly used currently.

The capture and immobilization of the assay reagent (complementary member of the binding pair) at the indicator zone can be accomplished by covalent bonding or, more commonly, by adsorption, such as by drying. Such capture also can be indirect, for example, by binding of latex beads coated with the reagent. Depending on the nature of the material comprising the lateral flow membrane, covalent bonding may be enabled, for example with use of glutaraldehyde or a carbodiimide. In immunoassays, most common binding pairs are antigen-antibody pairs; however, multiple other binding pairs can be performed, such as enzyme-substrate and receptor-ligand.

In some embodiments, the indicator zone further comprises a test line and a control line. A test line can comprise an immobilized binding reagent. When antibodies are used to develop a test line in the LFD that employs a sandwich type of assay, they are applied at a ratio of about 1-3 g/cm across the width of a strip 1 mm wide; hence, antibody concentration is about 10-30 g/cm², which is about 25-100 fold that used in an ELISA. Brown, M. C, Antibodies: key to a robust lateral flow immunoassay, in Lateral Flow Immunoassay, H. Y. T. R. C. Wong, Editor. 2009, Humana Press: New York, New York. p. 59-74.

Further, in some embodiments, the presently disclosed lateral flow assays can be used to detect multiple analytes in a sample. For example, in a lateral flow assay, the reagent zone can comprise multiple labeled reagents, each capable of binding to a different analyte in a liquid sample or a single labeled reagent capable of binding to multiple analytes. If multiple labeled reagents are used in a lateral flow assay, the reagents may be differentially labeled to distinguish different types of analytes in a liquid sample. It also is possible to place multiple lines of capture antibodies on the membrane to detect different analytes. Combinations of antibodies that detect different epitopes of an analyte may optimize specificity.

For quality control, typically a lateral flow membrane can include a control zone comprising a control line. The term “control zone” refers to a portion of the test device comprising a binding molecule configured to capture the labeled reagent. In a lateral flow assay, the control zone may be in liquid flow contact with the detection zone of the carrier, such that the labeled reagent is captured on the control line as the liquid sample is transported out of the detection zone by capillary action. Detection of the labeled reagent on the control line confirms that the assay is functioning for its intended purpose. Placement of a control line can be accomplished using a microprocessor controlled TLC spotter, in which a dispenser pump releases a constant volume of reagent across the membrane.

A typical lateral flow device can also comprise an absorbent pad. The absorbent pad comprises an “absorbent material,” which as used herein, refers to a porous material having an absorbing capacity sufficient to absorb substantially all the liquids of the assay reagents and any wash solutions and, optionally, to initiate capillary action and draw the assay liquids through the test device. Suitable absorbent materials include, for example, nitrocellulose, nitrocellulose blends with polyester or cellulose, untreated paper, porous paper, rayon, glass fiber, acrylonitrile copolymer, plastic, glass, or nylon.

In some embodiments, a lateral flow membrane is bound to one or more substantially fluid-impervious sheets, one on either side, e.g., a bottom sheet and a complimentary top sheet with one or more windows defining an application zone and an indicator zone. A typical lateral flow device also can include a housing. The term “housing” refers to any suitable enclosure for the presently disclosed lateral flow devices. Exemplary housings will be known to those skilled in the art. The housing can have, for example, a base portion and a lid portion. The lid portion can include a top wall and a substantially vertical side wall. A rim may project upwardly from the top wall and may further define a recess adapted to collect a sample from a subject. Suitable housings include those provided in U.S. Pat. No. 7,052,831 to Fletcher et al and those used in the BD Directigen™ EZ RSV lateral flow assay device.

In some embodiments, target analytes such as EPCAM, H4C5 and/or TTC3 can be measured in whole, unconcentrated, or otherwise unprocessed, biological samples using the presently disclosed methods and devices. In other embodiments, the biological sample can be processed, e.g., concentrated, diluted, filtered, and the like, prior to performing the test. The pre-treatment of a urine sample can include diluting the urine sample in an aqueous solution, concentrating the urine sample, filtering the urine sample, or a combination thereof.

One of ordinary skill in the art upon review of the presently disclosed subject matter would appreciate that the pre-treatment steps can be performed in any particular order, e.g., in some embodiments, the sample can be diluted or concentrated and then filtered, whereas in other embodiments, the sample can be filtered and then diluted or concentrated. In particular embodiments, the presently disclosed methods include filtering the urine sample, for example, through a desalting column, to remove a molecule that might interfere with the detection of antigen in the urine sample. This step can be performed with or without any further dilution or concentration of the sample.

Thus, in some embodiments, the lateral flow device further comprises an apparatus adapted to pre-treat the biological sample before contacting the biological sample with at least one antibody specific for EPCAM, at least one antibody specific for H4C5 and/or at least one antibody specific for TTC3. In particular embodiments, the apparatus is adapted to filter, dilute, or concentrate the biological sample, or combinations thereof. In an alternative embodiment, the apparatus can be adapted to remove an inhibitor that interferes with the detection of EPCAM and/or H4C5 in the biological sample, in particular, a urine sample.

In other embodiments, different parameters of the test, e.g., incubation time, can be manipulated to increase sensitivity and/or specificity of the test to eliminate the need for processing the biological sample.

### VI. Treatment Methods

In another aspect, the present invention provides a prostate cancer therapy or therapeutic interventions practically applied following the measurement/detection of biomarkers. In particular embodiments, therapeutic intervention comprises prostatectomy, radiation therapy, cryotherapy (also referred to as cryosurgery or cryoablation), hormone therapy, chemotherapy, immunotherapy and combinations thereof.

Prostatectomy includes radical prostatectomy (open (radical retropubic prostatectomy or radical perineal prostatectomy) or lateral (laparoscopic radical prostatectomy including robotic-assisted), and transurethral resection of the prostate (TURP).

Radiation therapy includes external beam radiation (three-dimensional conformal radiation therapy (3D-CRT), intensity modulated radiation therapy (IMRT), stereotactic body radiation therapy (SBRT), proton beam radiation therapy) and brachytherapy (internal radiation) (permanent (low dose rate or LDR) brachytherapy or temporary (high dose rate or HDR) brachytherapy).

Hormone therapy (androgen suppression therapy) includes orchiectomy (surgical castration), luteinizing hormone-release hormone (LHRH) agonists (e.g., leuprolide, goserelin, triptorelin, histrelin), LHRH antagonists (e.g., degarelix), treatment to lower androgen levels from the adrenal glands (e.g., abiraterone, ketoconazole), anti-androgens (e.g., flutamide, bicalutamide, nilutamide, enzalutamide, apalutamide), and estrogens.

Chemotherapy includes treatment with compounds including, but not limited to, docetaxel, cabazitaxel, mitoxantrone, and estramustine.

Immunotherapy includes, but is not limited to, a cancer vaccine (e.g., sipuleucel-T), as well as immune checkpoint inhibitors (e.g., PD-1 inhibitors including pembrolizumab). Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-O11 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

A prostate therapeutic intervention can comprise a targeted therapy including poly(ADP)-ribose polymerase (PARP) inhibitor (e.g., niraparib (zejula), olaparib (lynparza), and rucaparib (rubraca)).

Other therapeutic interventions for prostate cancer include an androgen receptor (AR)-targeted therapy (e.g., enzalutamide, ARN-509, ODM-201, EPI-001, hydrazinobenzoylcurcumin (HBC), aberaterone, geleterone, and seviteronel), an antimicrotubule agent, an alkylating agent and an anthracenedione.

In particular embodiments, a therapeutic intervention for prostate cancer can include the administration of drugs including, but not limited to, Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate), Zytiga (Abiraterone Acetate).

### VII. Kits

In another aspect, the present invention provides kits for detecting one or more biomarkers. The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of one or more biomarkers including by any one or more of mass spectrometry, antibody method, antibodies, lectins, nucleic acid aptamer method, nucleic acid aptamers, immunoassay, ELISA, immunoprecipitation, SISCAPA, Western blot, PCR (qPCR, digital PCR, etc.), lateral flow/dipstick or combinations thereof; and (b) reagents and instructions for sample processing, preparation and biomarker measurement/detection. The kit can further comprise (c) instructions for using the kit to measure biomarkers in a sample obtained from the subject.

In particular embodiments, the kit comprises reagents necessary for processing of samples and performance of an assay. In a specific embodiment, the assay is an immunoassay such as an ELISA. Thus, in certain embodiments, the kit comprises a substrate for performing the assay (e.g., a 96-well polystyrene plate). The substrate can be coated with antibodies specific for a biomarker protein. In a further embodiment, the kit can comprise a detection antibody including, for example, a polyclonal antibody specific for a biomarker protein conjugated to a detectable moiety or label (e.g., horseradish peroxidase). The kit can also comprise a standard, e.g., a human protein standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having protein biomarker capture reagents attached thereon. The kit may further comprise a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In a specific embodiment, a kit comprises one or more of (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker.

In certain embodiments, a subject can be diagnosed by adding a biological sample (e.g., urine) from the patient to the kit and detecting the relevant protein biomarkers conjugated with antibodies and/or lectins, specifically, by a method which comprises the steps of: (i) collecting serum from the patient; (ii) adding urine from patient to a diagnostic kit; and, (iii) detecting the protein biomarkers conjugated with antibodies/lectins. If the biomarkers are present in the sample, the antibodies/lectins will bind to the sample, or a portion thereof. In other kit and diagnostic embodiments, urine will not be collected from the patient (i.e., it is already collected). In other embodiments, the sample may comprise a urine, blood, plasma sweat, tissue, blood or a clinical sample.

The kit can also comprise a washing solution or instructions for making a washing solution, in which the combination of the capture reagents and the washing solution allows capture of the protein biomarkers on the solid support for subsequent detection by, e.g., antibodies/lectins or mass spectrometry. In a further embodiment, a kit can comprise instructions for suitable operational parameters in the form of a label or separate insert. For example, the instructions may inform a consumer about how to collect the sample, etc. In yet another embodiment, the kit can comprise one or more containers with protein biomarker samples, to be used as standard(s) for calibration or normalization. Detection of the markers described herein may be accomplished using a lateral flow assay.

In particular embodiments, the target proteins of the present invention can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling target protein enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

In certain embodiments, the kit comprises reagents and components necessary for performing an electrochemiluminescent ELISA.

certain embodiments, the kit comprises the use of a lateral flow apparatus, dipstick, assay stick with immunochromatographic detection display, and any such apparatus know to those skilled in the art. In certain embodiments, reagents and/or detection components may be immobilized on the apparatus itself (i.e., on the dipstick).

In some embodiments, the kit comprises a reagent that permits quantification of one or more of the nucleic acid markers described herein (eccDNA, mRNA, circRNA, lncRNA, etc.). In some embodiments, the kit comprises: (i) at least one reagent that allows quantification (e.g., determining the abundance, concentration or level) of an expression product of one or more of nucleic acid markers in a biological sample; and optionally (ii) instructions for using the at least one reagent. The kit can further comprise reagents for detection/measurement of other biomarkers.

A nucleic acid-based detection kit may include a primer or probe that specifically hybridizes to a target polynucleotide. The kit can further include a target biomarker polynucleotide to be used as a positive control. Also included may be enzymes suitable for amplifying nucleic acids including various polymerases (reverse transcriptase, Taq, Sequenase™, DNA ligase etc., depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

In a more specific embodiment, the kit is provided as a PCR kit comprising primers that specifically bind to one or more of the nucleic acid biomarkers described herein. The kit can further comprise substrates and other reagents necessary for conducting PCR (e.g., quantitative real-time PCR, digital PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having myocardial injury based on a comparison of the measured levels of one or more biomarkers to one or more reference controls.

The reagents described herein, which may be optionally associated with detectable labels, can be presented in the format of a microfluidics card, a chip or chamber, a microarray or a kit adapted for use with the assays described in the examples or below, e.g., RT-PCR, Q PCR, digital PCR techniques described herein.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

It is an object of the present invention to develop a sensitive, specific, and cost-effective non-invasive molecular diagnostic test to screen PCa patients for the detection of aggressive cancer from its indolent form. The present inventor has focused on identifying RNAs (coding and noncoding RNAs)¹⁻², disease specific SNPs³, metabolites and lipid^{2,4} markers in PCa urine and biopsy samples. The significant discovery to date in the clinic and the laboratory is the identification of a panel of urine-enriched RNAs and metabolites in prostate cancer patients. A PCa detection assay is developed using urine-specific RNAs (coding and noncoding), extracellular DNA (eccDNA) and metabolites⁴ in a statistically significant patient population. Although several biotechnology companies have developed FDA-approved PCa molecular markers (PSA, PCA3, etc.), these markers have failed to produce expected results. One of the major limitations of existing PCa markers is the reliance on a single biomarker (either PSA or PCA3). Cancer is recognized as a multistep process involving multiple genomic and epigenomic alterations in many phases. The complexity of cancer requires multivariate assays for accurate diagnosis, prognosis, and treatment monitoring. Multivariate gene expression assays have recently been proven to be feasible (Oncotype DX and MammaPrint for determining whether chemotherapy is necessary for breast cancer), but these tests are expensive and often need multiple biopsies and special specimens collection processes. A goal of the present invention is the development of an accurate and reproducible multivariate assay to detect the aggressive form of PCa in easily accessible tissue and urine samples. The major advantage of multiplex assays for clinical use is that they have the “power” to be highly accurate. The establishment of molecular assays that rival the accuracy of invasive procedures will shift clinical practice paradigms. The present inventor strongly believes that a urine-based combinatorial multianalyte (RNAs and eccDNA) assay is a powerful approach to detect aggressive PCa from its non-aggressive form.

Example 1: E3 ubiquitin-protein ligase, Tetratricopeptide Repeat Domain 3 (TTC3), H4 Clustered Histone 5 (H4C5), and epithelial cell adhesion molecule (EpCAM) are novel urine-enriched liquid biopsy biomarkers to detect prostate cancer in men.

Prostate cancer (PCa) is the most common cancer in men, with over 250,000 prostate cancer diagnoses per year in the United States (1). Although it is generally an indolent disease (low-grade, low-stage disease), PCa remains the second-leading cause of cancer death in men. In recent years, the combination of multiple treatment options (surgery, radiation, chemotherapy, androgen deprivation therapy) improved the median survival of PCa patients (2). While the incidence rate has decreased overall, the incidence in advanced-stage PCa has increased by 4% to 6% from 2014 to 2018. Moreover, the overall decrease in PCa incidence and mortality rate has been attributed to the widespread scrutiny of patient management (3). In clinical practice, biomarkers testing can better characterize tumor alterations. Prostate-specific antigen (PSA) is the most clinically accepted serum biomarker used for PCa, however, its specificity is limited because men with benign prostatic hypertrophy or prostatitis tends to perform high levels of PSA. The two prospective screening trials in the U.S. and Europe failed to demonstrate a concordant benefit in overall patient survival from PSA screening (4, 5). The combined application of imaging and biomarkers found in serum, urine, and tissue, have become increasingly matured in emerging clinical diagnosis (6). Hence, the identification of significant and reliable biomarkers associated with PCa detection and monitoring disease progression would be critical in guiding the clinical decision-making.

The ideal biomarker for clinical use should have three major characteristics: 1) a safe and easy means of measurement, preferably non-invasively; 2) high sensitivity, specificity, and positive and negative predictive values for its intended outcome; and 3) improves decision-making abilities in conjunction with clinicopathological parameters. Urine as a noninvasive and easily accessible biofluid, is emerging an essential source for biomarkers, especially in the early diagnosis and post-treatment monitoring of tumors (7). The present inventor has reported that integrated analysis of RNAs and metabolites obtained from urine samples of PCa patients and healthy individuals revealed abnormal gene signature subserve a distinction between PCa and normal individuals (8). Recently, some studies revolved around urine biomarker tests which help provide more specificity, including SelectMDx (DLX1, HOXC6) (9), ExoDx Prostate IntelliScore (EPI) (SPDEF, ERG, PCA3) (10), and Michigan Prostate Score (PCA3, PSA, TMPSS2:ERG) (11). These tests are endorsed by the National Comprehensive Cancer Network Guidelines, but the results are mixed (12).

Therefore, the present Example was designed to assess the diagnostic accuracy of urine multivariable biomarker for PCa, as part of the National Cancer Institute's Early Detection Research Network (EDRN)-defined phase II biomarker study (13). The assay is unique in that it, in some embodiments, does not require prior prostate examination and urine can be easily collected as part of a basic clinical workflow. Urine dipstick as a simple, cheap and rapid test are widely used as a screening and diagnostic tool for disease.

### Materials and Methods

Study population. The Human Research Ethics Committee and IRB protocols of the Johns Hopkins University School of Medicine approved the research for this study (Reference No. 237998). PCa patients were recruited at AdventHealth Global Robotics Institute, Celebration, FL, USA between September 2021 and July 2022. The initial diagnosis of PCa was based on trans-rectal ultrasound guided prostate biopsy and confirmed by histological and immunohistochemical examination of resected tissue. Urine was collected before and after the resection by robot-assisted radical prostatectomy. BPH patients were recruited through University of Florida, Gainesville, FL, USA. Normal controls were volunteers recruited through the John Hopkins University School of Medicine. Normal controls did not have a personal or family history of prostate cancer and did not have any significant lower urinary tract symptoms. Clinicopathological data were collected from the histopathology report of the prostate biopsies. Parameters collected included preoperative PSA, number of cores taken, number of cores positive for cancer, and overall ISUP Grade Group. Written consent forms were obtained in all cases. Patient demographics are displayed in Table 2.

Urine Sample Collection. First and midstream urine after waking up in the morning were collected. Urine samples were processed immediately by adding urine preservation solution (Norgen Bioteck) and kept at room temperature until centrifugation to separate the exfoliated cells in the urine samples. For the urine dipstick assay, three drops of fresh urine were used prior to further processing of a urine sample. The exfoliated cells from urine samples were used for total RNA purification using the miRNeasy mini kit (Qiagen). Total RNA was subjected to quantitative real-time PCR to identify gene expression. Cell-free urine was applied to ELISA assay.

ELISA. Soluble EpCAM levels in urine samples were measured using a human EpCAM DuoSet ELISA kit (R&D Systems) following manufacturer's instructions. Urine samples were vortexed at room temperature and centrifuged at 1000 g for 10 min. To each well of the assay were added 100 μl of urine supernatant, and seven-point calibration curves constructed using two-fold dilutions of 1 ng/ml standard. The optical density was determined at 450 nm (wavelength correction at 570 nm) using a EnVision 2105 microplate reader (PerkinElmer). The EpCAM concentrations (pg/ml) were obtained with a two-parameter logistic curve, fitted for the standard value and multiplied by the dilution factor. All measurements were done in duplicate.

RNA extraction, reverse transcription, preamplification and quantitative real-time PCR of exfoliated cells. Total RNA of exfoliated cells from urine samples were extracted using QIAzol lysis reagent and miRNeasy mini kit (Qiagen). The samples were treated with DNase I (Qiagen) and RNA concentrations were measured using NanoDrop 8000 (Thermo Scientific). cDNA was synthesized using the high-capacity cDNA reverse transcription kit (Applied Biosystems) according to the manufacturer's instructions. The total volume of pre-amplification was 50 μl for each sample. The reaction contained 25 μl of pre-amplification mastermix, 24 μl of cDNA, 1 μl of pooled primers with a final concentration of each primer of 10 nM. A 14-cycle cDNA pre-amplification was then performed according to the following schedule: 95° C. for 10 min, 95° C. for 15 s and 58° C. for 4 min. Gene expression was quantified using SYBR Green Master Mix (Applied Biosystems). 3-actin was used as an internal control. Quantitative real-time PCR was performed on QuantStudio 5 (Applied Biosystems). Relative changes of gene expression were analyzed using 2^{-ΔCT} method.

Cell culture. PCa cell lines PC3 and LNCaP were obtained from the American Type Culture Collection (ATCC), and the cells were cultured in Ham's F-12K Medium and RPMI 1640 medium (Gibco), respectively, with 10% FBS and 1% penicillin/streptomycin. The cell lines were authenticated by STR profiling and regularly tested for mycoplasma contamination throughout the study.

Cell transfection. Cells were transiently transfected with silencer select siRNAs for EpCAM (s8370, s8371, s8372) or TTC3 (s14475, s14476, s14477) or silencer select negative control #1 siRNA (Invitrogen) at 20 nM. Transfection was performed using lipofectamine RNAiMAX reagents (Invitrogen). Assays were performed 48 h (for qPCR) or 72 h (for western blot) after transfection unless otherwise stated.

Cell proliferation assay. Cells were seeded in 96-well plates at 3000 cells per well. EpCAM and TTC3 siRNAs were added the following day. After 24, 48, 72 hours of incubation, cell proliferation was measured by cellTiter 96 AQueous non-radioactive cell proliferation assay (Promega). The absorbance value was measured at 490 nm using a EnVision 2105 microplate reader (PerkinElmer).

In this study, voided urine (50 ml) from pre and post-prostatectomy men with PCa was used and urine from normal healthy men was used as control. RNA from exfoliated cells and debris shed into urine was isolated and RNA-sequencing was performed using the Illumina Next-seq 550 platform. Advanced computational and machine-learning approaches were employed to identify candidate biomarkers in men with PCa. The TCGA database was examined to validate the PCa-specific expression of the identified RNA in tumor tissues. Two RNA markers were further tested by qPCR, and one urinary soluble protein marker was measured by immunoassays.

The study included 106 men with PCa and 88 control men. The presence of ≥1 RNA markers (TTC3, H4C5) and a protein marker (EpCAM) were identified and validated in urine as potential candidate biomarkers for PCa detection. These markers were tested and developed using qPCR for TTC3, H4C5, and ELISA assay for EpCAM with higher specificity and sensitivity (Table 1). The results outperformed known urinary markers, PCA3 and SPDEF (FIG. 31). TTC3, H4C5, and EpCAM markers diminished to low or undetectable levels in post-prostatectomy compared to pre-prostatectomy men with PCa. shRNA knockdown of TTC3 and EpCAM in androgen-sensitive and insensitive cells induced biological changes, suggesting their relevance to Prostate Cancer.

Thus, in particular embodiments, the present invention provides a highly accurate panel of 3 urine-based biomarkers that detect PCa comprising EpCAM (protein), and TTC3 and H4C5 (RNA). In certain embodiments, a urine liquid biopsy biomarkers assay comprises a highly accurate panel of 3 urine-based biomarkers assay that discriminates between PCa and healthy men. The assay was associated with improved identification of patients with higher-grade prostate cancer among men with elevated PSA levels and could reduce the total number of unnecessary biopsies.

### REFERENCES

1. Siegel R L, Miller K D, Fuchs H E, Jemal A. Cancer statistics, 2022. CA Cancer J Clin. 2022 January; 72(1):7-33.
2. Litwin M S, Tan H J. The Diagnosis and Treatment of Prostate Cancer: A Review. JAMA. 2017 Jun. 27; 317(24):2532-2542.
3. Gulati R, Tsodikov A, Etzioni R, Hunter-Merrill R A, Gore J L, Mariotto A B, Cooperberg M R. Expected population impacts of discontinued prostate-specific antigen screening. Cancer. 2014 Nov. 15; 120(22):3519-26.
4. Andriole G L, Crawford E D, Grubb R L 3rd, Buys S S, Chia D. Church T R. Fouad M N, Gelmann E P, Kvale P A, Reding D J, Weissfeld J L, Yokochi L A, O'Brien B. Clapp J D, Rathmell J M, Riley T L, Hayes R B, Kramer B S, Izrmirlian G, Miller A B, Pinsky P F, Prorok P C, Gohagan J K, Berg C D; PLCO Project Team. Mortality results from a randomized prostate-cancer screening trial. N Engl J Med. 2009 Mar. 26; 360(13):1310-9.
5. Schruder F H, Hugosson J, Roobol M J, Tammela T L, Ciatto S, Nelen V, Kwiatkowski M, Lujan M, Lilja H, Zappa M, Denis L J, Recker F, Berenguer A, Määttänen L, Bangma C H, Aus G, Villers A, Rebillard X, van der Kwast T, Blijenberg B G, Moss S M, de Koning H J. Auvinen A; ERSPC Investigators. Screening and prostate-cancer mortality in a randomized European study. N Engl J Med. 2009 Mar. 26; 360(13):1320-8.
6. Cucchiara V, Cooperberg M R, Dall'Era M, Lin D W, Montorsi F, Schalken J A, Evans C P. Genomic Markers in Prostate Cancer Decision Making. Eur Urol. 2018 April; 73(4):572-582.
7. Bax C, Lotesoriere B J, Sironi S, Capelli L. Review and Comparison of Cancer Biomarker Trends in Urine as a Basis for New Diagnostic Pathways. Cancers (Basel). 2019 Aug. 25; 11(9):1244.
8. Lee B, Mahmud I, Marchica J, Derezinski P, Qi F, Wang F, Joshi P, Valerio F, Rivera I, Patel V, Pavlovich C P, Garrett T J, Schroth G P, Sun Y, Perera R J. Integrated RNA and metabolite profiling of urine liquid biopsies for prostate cancer biomarker discovery. Sci Rep. 2020 Feb. 28; 10(l):3716.
9. Van Neste L, Hendriks R J, Dijkstra S, Trooskens G. Cornel E B, Jannink S A, de Jong H, Hessels D, Smit F P, Melchers W J, Leyten G H, de Reijke T M, Vergunst H, Kil P, Knipscheer B C, Hulsbergen-van de Kaa C A, Mulders P F, van Oort I M, Van Criekinge W, Schalken J A. Detection of High-grade Prostate Cancer Using a Urinary Molecular Biomarker-Based Risk Score. Eur Urol. 2016 November; 70(5):740-748.
10. McKiernan J, Donovan M J, O'Neill V, Bentink S, Noerholm M, Belzer S, Skog J, Kattan M W, Partin A, Andriole G, Brown G, Wei J T, Thompson I M Jr, Carroll P. A Novel Urine Exosome Gene Expression Assay to Predict High-grade Prostate Cancer at Initial Biopsy. JAMA Oncol. 2016 Jul. 1; 2(7):882-9.
11. Sanda M G, Feng Z, Howard D H, Tomlins S A, Sokoll L J, Chan D W, Regan M M, Groskopf J, Chipman J, Patil D H, Salami S S, Scherr D S, Kagan J, Srivastava S, Thompson I M Jr, Siddiqui J, Fan J, Joon A Y, Bantis L E, Rubin M A, Chinnayian A M., Wei J T; and the EDRN-PCA3 Study Group, Bidair M, Kibel A, Lin D W, Lotan Y, Partin A, Taneja S. Association Between Combined TMPRSS2:ERG and PCA3 RNA Urinary Testing and Detection of Aggressive Prostate Cancer. JAMA Oncol. 2017 Aug. 1; 3(8):1085-1093.
12. Carroll P H, Mohler J L. NCCN Guidelines Updates: Prostate Cancer and Prostate Cancer Early Detection. J Natl Compr Cane Netw. 2018 May; 16(5S):620-623.
13. Pepe M S, Etzioni R, Feng Z, Potter J D, Thompson M L, Thornquist M, Winget M, Yasui Y. Phases of biomarker development for early detection of cancer. J Natl Cancer Inst. 2001 Jul. 18; 93(14):1054-61,

TABLE 1
Urinary biomarker performance data for the diagnosis of PCa.

| | Optimal | AUC | Sensitivity | Specificity | | |
|---|---|---|---|---|---|---|
| Biomarker | Cutpoint¹ | (95% CI) | (95% CI) | (95% CI) | PPV | NPV |
| EpCAM (ELISA) | 26.86 | 0.99 | 0.94 | 0.99 | 0.99 | 0.94 |
| | | (0.99-1.00) | (0.88, 0.98) | (0.94, 1.00) | | |
| H4C5 (qPCR) | 1068.32 | 0.96 | 0.88 | 0.94 | 0.93 | 0.88 |
| | | (0.93-0.98) | (0.80, 0.94) | (0.87, 0.98) | | |
| TTC3 (qPCR) | 69.92 | 0.92 | 0.89 | 0.83 | 0.85 | 0.87 |
| | | (0.89-0.95) | (0.82, 0.94) | (0.74, 0.90) | | |

¹Optimal outpoints were determined using the Youden index.
AUC, area under the curve; PPV, Positive predictive value; NPV, Negative predictive value.

TABLE 2
Demographic and clinicopathologic
characteristics of 107-subject study cohort.

| | | Median |
|---|---|---|
| Characteristics | Counts | (range) or n % |
| Age (years) | 107 | 65 (40-80) |
| Pre-biopsy serum | 107 | 6.6 (0.21-37.13) |
| tPSA level (ng/ml) | | |
| Race | | |
| Caucasian | 90 | 84.1% |
| African-American | 10 | 9.3% |
| Native-American | 2 | 1.9% |
| Asian or Pacific Islander | 2 | 1.9% |
| Other | 3 | 2.8% |
| Suspicious DRE | 107 | 100% |
| Family history of PCa | 39/107 | 36.4% |
| Family history of BCa | 22/107 | 20.6% |
| Biopsy result | | |
| Positive | 22/107 | 20.6% |
| Negative | 85/107 | 79.4% |
| Clinical Stage | | |
| cT1-2a | 101 | 94.4% |
| cT2b-c | 6 | 5.6% |
| Gleason score | | |
| GS = 6 (3 + 3) | 13 | 12.1% |
| GS = 7 (3 + 4) | 47 | 43.9% |
| GS = 7 (4 + 3) | 18 | 16.8% |
| GS = 8 (4 + 4) | 19 | 17.8% |
| GS = 9 (4 + 5 or 5 + 4) | 10 | 9.4% |

Abbreviations:
DRE, digital rectal examination;
PCa, prostate cancer;
BCa, Breast cancer;
GS, Gleason score.

TABLE 3
Sensitivity and the Specificity of Biomarkers

| | Sensitivity | Specificity | AUC |
|---|---|---|---|
| Biomarker | (95% CI) | (95% CI) | (95% CI) |
| EpCAM | 0.94 (0.88, 0.98) | 0.99 (0.94, 1.00) | 0.99 (0.99-1.00) |
| (ELISA) | | | |
| TTC3 | 0.89 (0.82, 0.94) | 0.83 (0.74, 0.90) | 0.92 (0.89-0.95) |
| H4C5 | 0.88 (0.80, 0.94) | 0.94 (0.87, 0.98) | 0.96 (0.93-0.98) |
| PCA3 | 0.71 (0.61, 0.79) | 0.83 (0.74, 0.90) | 0.81 (0.76-0.86) |
| SPDEF | 0.56 (0.46, 0.66) | 0.88 (0.80, 0.94) | 0.78 (0.72-0.83) |

TABLE 4
Clinical significance of EPCAM protein, TTC3 and
H4C5 RNA expression in 107 patients with PCa.

| | | EPCAM | | TTC3 | | H4C5 | |
|---|---|---|---|---|---|---|---|
| | | (Protein) | p | (RNA) | p | (RNA) | p |
| | n | Mean ± SD | Value | Mean ± SD | Value | Mean ± SD | Value |
| Age (years) | | | 0.93 | | 0.87 | | 0.98 |
| <65 | 53 | 58.48 ± 38.92 | | 0.14 ± 0.07 | | 2.33 ± 1.17 | |
| ≥65 | 54 | 57.88 ± 32.46 | | 0.14 ± 0.08 | | 2.34 ± 1.30 | |
| Race | | | 0.49 | | 0.78 | | 0.09 |
| Caucasian | 90 | 59.63 ± 36.19 | | 0.14 ± 0.08 | | 2.22 ± 1.15 | |
| African-American | 10 | 55.70 ± 41.28 | | 0.15 ± 0.05 | | 2.83 ± 1.06 | |
| Other | 7 | 43.25 ± 13.92 | | 0.13 ± 0.04 | | 3.09 ± 2.01 | |
| tPSA level (ng/ml) | | | 0.21 | | 0.86 | | 0.87 |
| <4 | 11 | 49.02 ± 18.20 | | 0.14 ± 0.06 | | 2.15 ± 0.92 | |
| 4-10 | 77 | 62.01 ± 39.49 | | 0.14 ± 0.07 | | 2.36 ± 1.23 | |
| ≥10 | 19 | 48.15 ± 22.85 | | 0.15 ± 0.08 | | 2.34 ± 1.44 | |
| Gleason score | | | 0.49 | | 0.70 | | 0.26 |
| Low (GS 6 + 7) | 78 | 59.67 ± 34.06 | | 0.14 ± 0.07 | | 2.25 ± 1.18 | |
| High (GS 8 + 9) | 29 | 54.23 ± 40.00 | | 0.13 ± 0.07 | | 2.56 ± 1.37 | |
| Clinical Stage | | | 0.42 | | 0.86 | | 0.67 |
| cT1-2a | 101 | 57.48 ± 33.64 | | 0.14 ± 0.07 | | 2.35 ± 1.25 | |
| cT2b-c | 6 | 69.75 ± 64.32 | | 0.13 ± 0.09 | | 2.12 ± 0.93 | |

TABLE 5
Identification and quantitative real-time PCR (qPCR) validation of differentially expressed
genes (DEGs) in prostate cancer from The Cancer Genome Atlas (TCGA) in PCa urine.

| | TCGA | | | | | qPCR | | | | |
|---|---|---|---|---|---|---|---|---|---|---|
| | | P. | adj. P. | | | | P. | Normal | PCa | Dysregulated |
| Gene | logFC | value | Val | Normal | PCa | FC | value | (2^{-ΔCt}) | (2^{-ΔCt}) | in PCa |
| COX20 | 2.189 | 0.002 | 0.034 | 86.576 | 131.897 | 0.087 | 0.024 | 0.3789 | 0.0335 | Down |
| TTC3 | 2.042 | 0.000 | 0.017 | 232.753 | 414.633 | 3.470 | 0.000 | 0.0254 | 0.0884 | Up |
| ZNF91 | 2.569 | 0.000 | 0.021 | 1075.505 | 1781.264 | 1.160 | 0.600 | 3.0314 | 3.4822 | Up |
| CAPN3 | 2.422 | 0.000 | 0.018 | 181.165 | 444.033 | 0.157 | 0.052 | 0.7579 | 0.1250 | Down |
| CANX | 2.112 | 0.001 | 0.029 | 770.684 | 1927.51 | 0.327 | 0.059 | 0.2500 | 0.0825 | Down |
| PBLD | 2.423 | 0.002 | 0.031 | 61.104 | 140.345 | 0.763 | 0.210 | 0.0136 | 0.0103 | Down |
| H4C2 | 2.789 | 0.000 | 0.017 | 51.132 | 110.941 | 5.228 | 0.002 | 0.0718 | 0.3789 | Up |
| EEF1G | 3.251 | 0.001 | 0.023 | 1847.119 | 4196.47 | 2.682 | 0.007 | 0.1895 | 0.5000 | Up |
| TOM1L1 | 2.117 | 0.000 | 0.016 | 101.631 | 168.382 | 359.686 | 0.002 | 0.0002 | 0.0670 | Up |
| LUC7L3 | 2.399 | 0.001 | 0.022 | 307.665 | 996.918 | 0.004 | 0.010 | 0.0312 | 0.0001 | Down |
| H4C3 | 2.864 | 0.000 | 0.017 | 44.676 | 89.39 | 2.185 | 0.018 | 0.3536 | 0.7579 | Up |
| ELK4 | 2.628 | 0.000 | 0.011 | 142.133 | 213.239 | 1.294 | 0.134 | 0.1895 | 0.2500 | Up |
| HMGN2P5 | 2.964 | 0.000 | 0.015 | 27.449 | 72.667 | 0.000 | 0.001 | 0.5743 | 0.0001 | Down |
| H1-4 | 2.589 | 0.001 | 0.023 | 88.499 | 209.691 | 2.941 | 0.026 | 0.0292 | 0.0884 | Up |
| OST4 | 2.738 | 0.001 | 0.021 | 38.678 | 72.828 | 1.204 | 0.124 | 0.7071 | 0.8706 | Up |
| SNURF | 2.960 | 0.000 | 0.019 | 109.93 | 237.095 | 0.028 | 0.014 | 1.1487 | 0.0335 | Down |
| PNPO | 2.234 | 0.000 | 0.019 | 86.072 | 153.821 | 0.014 | 0.002 | 0.0385 | 0.0005 | Down |
| NUDT4 | 2.886 | 0.000 | 0.016 | 33.491 | 93.553 | 0.042 | 0.002 | 1.1487 | 0.0474 | Down |
| AK4 | 2.525 | 0.001 | 0.027 | 31.168 | 66.824 | 0.008 | 0.000 | 0.0110 | 0.0001 | Down |
| RSL1D1 | 2.456 | 0.000 | 0.017 | 117.136 | 220.488 | 0.008 | 0.004 | 4.0000 | 0.0292 | Down |
| H4C5 | 2.608 | 0.002 | 0.037 | 198.317 | 386.684 | 3.383 | 0.015 | 0.8706 | 2.8284 | Up |
| UGDH | 2.415 | 0.000 | 0.014 | 94.203 | 156.088 | 0.858 | 0.360 | 0.0718 | 0.0583 | Down |
| RIDA | 2.726 | 0.002 | 0.033 | 121.201 | 250.349 | 6.021 | 0.012 | 0.0272 | 0.1649 | Up |
| TRAPPC5 | 3.005 | 0.000 | 0.021 | 19.724 | 43.869 | 0.136 | 0.098 | 0.1649 | 0.0221 | Down |
| ZNF181 | 2.307 | 0.000 | 0.011 | 50.729 | 98.175 | 0.074 | 0.000 | 0.0508 | 0.0036 | Down |
| NPM1 | 3.407 | 0.000 | 0.019 | 830.985 | 2052.852 | 0.529 | 0.018 | 21.1121 | 11.3137 | Down |
| PTMA | 2.496 | 0.001 | 0.028 | 1183.807 | 2869.023 | 0.163 | 0.019 | 45.2548 | 7.4643 | Down |
| VDAC1 | 2.490 | 0.001 | 0.028 | 135.66 | 276.564 | 0.066 | 0.008 | 0.0078 | 0.0005 | Down |
| HSPD1 | 2.375 | 0.002 | 0.037 | 801.075 | 1475.234 | 0.818 | 0.476 | 0.1436 | 0.1166 | Down |
| HSPE1 | 2.478 | 0.004 | 0.049 | 110.215 | 157.201 | 0.128 | 0.004 | 6.4980 | 0.8123 | Down |
| NIT2 | 2.062 | 0.002 | 0.032 | 69.752 | 123.563 | 0.000 | 0.000 | 3.0314 | 0.0000 | Down |
| RBIS | 3.001 | 0.000 | 0.013 | 26.038 | 61.975 | 0.762 | 0.420 | 1.2311 | 0.9330 | Down |
| COX6C | 2.206 | 0.001 | 0.029 | 24.068 | 38.012 | 0.000 | 0.001 | 0.4353 | 0.0002 | Down |
| MRPS21 | 3.444 | 0.000 | 0.011 | 59.437 | 131.776 | 1.084 | 0.433 | 2.8284 | 3.2490 | Up |
| ODC1 | 2.413 | 0.000 | 0.017 | 90.795 | 167.905 | 0.064 | 0.000 | 2.2974 | 0.1436 | Down |
| NCALD | 2.230 | 0.002 | 0.034 | 21.948 | 96.4 | 4.494 | 0.046 | 0.0002 | 0.0009 | Up |
| DDAH1 | 2.142 | 0.003 | 0.041 | 41.515 | 54.443 | 0.089 | 0.004 | 0.3299 | 0.0313 | Down |
| NDUFB9 | 2.817 | 0.002 | 0.035 | 190.83 | 339.838 | 2.320 | 0.023 | 0.4353 | 1.0000 | Up |
| MRPL51 | 2.089 | 0.000 | 0.019 | 29.281 | 52.181 | 0.140 | 0.000 | 6.0629 | 0.8123 | Down |
| GATD3B | 2.417 | 0.001 | 0.027 | 32.594 | 60.388 | 0.834 | 0.628 | 0.0625 | 0.0508 | Down |
| COA4 | 2.282 | 0.001 | 0.024 | 6.933 | 10.502 | 0.896 | 0.606 | 0.1436 | 0.1340 | Down |
| ATP5MC1 | 2.104 | 0.001 | 0.023 | 34.757 | 65.533 | 0.151 | 0.036 | 0.2333 | 0.0335 | Down |
| MAP7 | 2.343 | 0.000 | 0.017 | 415.075 | 347.456 | 0.492 | 0.001 | 0.2333 | 0.1088 | Down |
| HOMER2 | 2.545 | 0.000 | 0.008 | 26.991 | 58.107 | 0.075 | 0.105 | 0.4061 | 0.0313 | Down |
| NFIX | 2.114 | 0.000 | 0.018 | 65.769 | 118.408 | 0.184 | 0.000 | 0.0002 | 0.0000 | Down |
| CCDC58 | 2.315 | 0.001 | 0.021 | 33.402 | 66.627 | 0.605 | 0.145 | 0.3078 | 0.1895 | Down |
| RAN | 2.234 | 0.001 | 0.029 | 82.955 | 121.073 | 2.494 | 0.034 | 0.6598 | 1.6245 | Up |
| EPCAM | 2.069 | 0.002 | 0.034 | 213.414 | 337.64 | 696.495 | 0.007 | 0.0002 | 0.1088 | Up |
| COX5A | 2.320 | 0.001 | 0.024 | 40.568 | 62.53 | 0.161 | 0.012 | 3.0314 | 0.4665 | Down |
| TMEM263 | 2.002 | 0.001 | 0.023 | 9.672 | 21.187 | 19.090 | 0.000 | 0.0002 | 0.0030 | Up |

EXAMPLE 2: Apply a group of urine-enriched RNAs (coding and noncoding) as PCa biomarkers. PCa is a leading cause of cancer death among men in the United States, with more than 3.6 million men living with prostate cancer. However, many newly diagnosed prostate cancer is indolent and clinically insignificant with low metastatic potential. Therefore, developing non-invasive and accurate markers for early detection to distinguish indolent cancers from aggressive is timely. Based on published^{1, 2, 4} and preliminary results, a panel of urine enriched RNAs (mRNAs, lncRNAs, and circRNAs) are potential biomarkers for PCa detection. A PCa-upregulated RNA panel (mRNAs, circular RNAs and long noncoding RNAs) and eccDNA are measured by qPCR and digital PCR in samples from statistically significant numbers, given the power-requirement, of patient cohorts: (a) urine samples from high-grade and low-grade PCa patients, (b) urine samples from non-cancerous but PSA elevated individuals (i.e., Benign Prostate Hyperplasia, Prostatitis, etc.) and (c) urine samples from control healthy individuals. The panel of RNA signatures is useful in establishing a novel PCa non-invasive test. The present inventors has developed ELISA-based methods to test these markers in the clinic.

EXAMPLE 3: Develop a multivariate logistic regression model to integrate PCa-specific RNA signatures to identify a multivariant biomarker test. The present inventor has identified a group of PCa-specific RNAs in urine compared to normal healthy individuals. RNA data have been and will be integrated with comprehensive gene expression analyses to interrogate complex gene networks for better PCa diagnosis. A multivariate logistic regression model is developed as a predictor of PCa and would be powerful for PCa detection in men and superior to single-molecule detection. Multianalyte markers (mRNAs, circRNAs, lncRNAs and eccDNAs) are applied in patient samples.

The major impact and the innovative aspect of the present invention is based on the non-invasive nature of multiple RNAs panels to detect aggressive PCa that cannot be done with current single biomarker tests (PCA3 or PSA). The present invention is supplemented with additional candidates as they become available for further enhance performance. A combinatorial “multi-RNA”-based molecular marker panel is developed.

All proposed makers could be tested in free-flow urine and not necessary for a prostate massage (example PCA3 testing).

### REFERENCES

1. Lee B, Mazar J, Aftab M N, Qi F, Shelley J, Li J L, Govindarajan S, Valerio F, Rivera I, Thum T, Tran T A, Kameh D, Patel V and Perera R J. Long noncoding RNAs as putative biomarkers for prostate cancer detection. J Mol Diagn. 2014; 16:615-26.
2. Mouraviev V, Lee B, Patel V, Albala D, Johansen T E, Partin A, Ross A and Perera R J. Clinical prospects of long noncoding RNAs as novel biomarkers and therapeutic targets in prostate cancer. Prostate Cancer Prostatic Dis. 2016; 19:14-20.
3. Lee B, Li J L, Marchica J, Mercola M, Patel V and Perera R J. Mapping genetic variability in mature miRNAs and miRNA binding sites in prostate cancer. J Hum Genet. 2021.
4. Lee B, Mahmud I, Marchica J, Derezinski P, Qi F, Wang F, Joshi P, Valerio F, Rivera I, Patel V, Pavlovich C P, Garrett T J, Schroth G P, Sun Y and Perera R J. Integrated RNA and metabolite profiling of urine liquid biopsies for prostate cancer biomarker discovery. Sci Rep. 2020; 10:3716.

1. A method for identifying a patient as having aggressive prostate cancer comprising the step of detecting overexpression relative to a control of epithelial cell adhesion molecule (EpCAM), H4 clustered histone 5 (H4C5), and tetratricopeptide repeat domain 3 (TTC3) in a sample obtained from the patient.

2. The method of claim 1, wherein the detecting step comprises detecting protein level of EpCAM in a urine sample.

3. The method of claim 1, wherein the detecting step comprises detecting ribonucleic acid (RNA) level of H4C5 and TTC3.

4. The method of claim 3, wherein the detecting step is performed using polymerase chain reaction (PCR).

5. The method of claim 1, wherein the method distinguishes among aggressive prostate cancer, indolent prostate cancer, benign prostate hyperplasia and prostatitis.

6. The method of claim 1, further comprising detecting overexpression relative to a control of one or more of messenger ribonucleic acid (mRNA), circulating RNA (circRNA), extracellular DNA and long non-coding RNA (lncRNA).

7. The method of claim 6, wherein the mRNA comprises one or more of RIDA, H1-4, H4C2 and H4C3.

8. The method of claim 6, wherein the circRNA comprises one or more of circ842, circ3266, circ1809, circ1979, circ645 and circ1607.

9. The method of claim 6, wherein the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

10. The method of claim 6, wherein the lncRNA comprises lnc-CCDC125-13 and/or ZNF667-AS1.

11. The method of claim 1, further comprising detecting one or more metabolites selected from the group consisting of asparagine, aspartate, glycerate, citrate, isocitrate, glutamate, itaconate, malate, meglutol, cis-aconitate, isoleucine, leucine, pantothenate, glutamine, nicotinate, threonine, ketoglutarate, alpha-ketoisovaleric acid (KIVA), cysteine, 3P glycerate, xanthine and hypoxanthine.

12. The method of claim 1, further comprising the step of administering a prostate cancer therapy to the patient identified as having aggressive prostate cancer.

13. The method of claim 12, wherein the prostate cancer therapy comprises prostatectomy, radiation therapy, cryotherapy, hormone therapy, chemotherapy, immunotherapy and combinations thereof.

14. A method for treating a patient having aggressive prostate cancer comprising the step of administering a prostate cancer therapy to a patient identified as having overexpression of EPCAM, H4C5 and TTC3 in a sample relative to a control.

15. The method of claim 14, wherein the patient sample further comprises overexpression relative to a control of one or more of mRNA, circRNA, eccDNA and lncRNA.

16. The method of claim 15, wherein the mRNA comprises one or more of RIDA, H1-4, H4C2 and H4C3.

17. The method of claim 15, wherein the circRNA comprises one or more of circ842, circ3266, circ1809, circ1979, circ645 and circ1607.

18. The method of claim 15, wherein the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

19. The method of claim 15, wherein the lncRNA comprises lnc-CCDC125-13 and/or ZNF667-AS1.

20. A method for identifying a patient as having aggressive prostate cancer comprising the step of detecting the overexpression of one or more of protein, mRNA, circRNA, eccDNA lncRNA in a sample obtained from the patient relative to a control.

21. The method of claim 20, wherein the protein comprises EPCAM.

22. The method of claim 20, wherein the mRNA comprises one or more of H4C5, TTC3, RIDA, H1-4, H4C2 and H4C3.

23. The method of claim 20, wherein the circRNA comprises one or more of circ842, circ3266, circ1809, circ1979, circ645 and circ1607.

24. The method of claim 20, wherein the eccDNA comprises one or more of chr22:50276214-50276428; chr20:2236337-2236458; chr6:54059859-54063911; chr16:85975027-85975617; chr3:5565190-5565271; chr10:130300872-130301712; chr11:58900903-59058535; chr22:44599233-49967822; chr17:69961543-69961943; chr18:9809075-9809266; chr17:80024303-80024653; chrY:10945178-11295108; chr7:65038315-65873352; chr1:21669328-93846973; chr6:168914322-168914396; chr6:35786783-35799011; chr6:26305559-28597426; chr9:34681483-34681981; chr1:207698916-207868701; chr8:57203766-57210492; chr16:20504588-20504731; chr3:67362279-136250669; chr1:248404181-248404245; chr12:1574344-1574491; chr7:66728585-73967789; chr1:55002895-55003057; and chr16:89907819-89908811.

25. The method of claim 20, wherein the lncRNA comprises lnc-CCDC125-13 and/or ZNF667-AS1.

26. The method of claim 20, wherein the detecting step comprises a lateral flow assay.

27. The method of claim 26, wherein the lateral flow device comprises a dipstick assay.

28. The method of claim 27, wherein the sample comprises free-flow urine and/or prostate massaged urine.

29. The method of claim 22, wherein the sample comprises blood or serum.
